(19) United States
(12) Patent Application Publication
(10) Pub. No.: US 2015/0209428 A1
(43) Pub. Date: Jul. 30, 2015

(54) PHARMACEUTICAL COMPOSITIONS FOR TOPICAL DELIVERY OF PHOTOSENSITIZERS AND USES THEREOF

(71) Applicant: DERMIRA, INC., Redwood City, CA (US)

(72) Inventors: Delphine Caroline Imbert, Cupertino, CA (US); David William Carey Hunt, Surrey (CA)

(21) Appl. No.: 14/423,041

(22) PCT Filed: Jul. 11, 2013
(86) PCT No.: PCT/US2013/050070

§ 371 Date: Feb. 20, 2015

Related U.S. Application Data
(60) Provisional application No. 61/670,554, filed on Jul. 11, 2012.
(60) Provisional application No. 61/706,732, filed on Sep. 27, 2012.
(60) Provisional application No. 61/708,845, filed on Oct. 2, 2012.

(51) Int. Cl. A61K 47/10; A61K 47/08; A61K 31/409

(52) U.S. Cl. CPC A61K 47/10; A61K 31/409; A61K 47/08; A61N 5/062

(57) ABSTRACT

The present disclosure includes and provides compositions comprising photosensitizing agents and their use in photodynamic therapy for the treatment of dermatological conditions.

### CROSS-REFERENCE TO RELATED APPLICATIONS

This application claims the benefit of U.S. Provisional Applications Ser. No. 61/670,554, filed Jul. 11, 2012, 61/706,732, filed Sep. 27, 2012, and 61/708,845, filed Oct. 2, 2012, the contents of which are incorporated in their entirety.

### FIELD OF THE INVENTION

The present disclosure includes and provides compositions comprising photosensitizing agents and their use in photodynamic therapy for the treatment of dermatological conditions.

### BACKGROUND OF THE INVENTION

Photodynamic therapy (PDT) is a procedure that uses light-activated drugs (photosensitizers) to treat a wide range of medical conditions. Accumulation of the photosensitizer in a target tissue that can be directly illuminated makes PDT a selective treatment. When a photosensitizer is activated by light, singlet oxygen and other free radicals are produced in tissues that have retained the drug. The interaction of these reactive oxygen species with biological macromolecules induces a cascade of biochemical reactions that cause changes in cell metabolism, and at high doses of drug and/or light, can result in cell death.

Photodynamic therapy (PDT) has been proposed as a treatment for a number of skin conditions, including acne vulgaris, hyperactive sebaceous glands, psoriasis, atopic dermatitis, and certain types of skin cancers. One of the challenges in performing PDT for these conditions has been targeting sufficient quantities of photosensitizer to the desired location in the skin without causing generalized and unwanted skin photosensitivity reactions such as erythema, pain, burning and itching after irradiation with light. For example, in treating conditions such as acne vulgaris, sebaceous gland hyperplasia, seborrhea and seborrheic dermatitis, conditions characterized by sebaceous gland hyperactivity, it would be desirable to have the photosensitizer drug selectively localized in the sebaceous glands.

A number of topical formulations of photosensitizers have been proposed for treating skin conditions (see for example, WO 2005/074987). Formulation composition may markedly influence topical photosensitizer delivery into the skin as well as skin appendages such as pilosebaceous units (PSU), structures consisting of a hair follicle with associated sebaceous glands. There is a need for better formulations that effectively deliver photosensitizer drugs into sebaceous glands.

### SUMMARY OF THE INVENTION

In one aspect, this invention provides a pharmaceutical composition useful for localizing a photosensitizer to a sebaceous gland, comprising a constituted formulation of a photosensitizing component comprising a photosensitizer supersaturated at ambient temperature, one or more solvents, and diethylene glycol monoethyl ether (DGME), wherein the photosensitizer is a green porphyrin present at a final concentration (w/w) of between about 0.1% to about 0.4% in the pharmaceutical composition; and wherein the one or more solvents comprise benzyl alcohol present at a final concentration (w/w) of between about 5% and about 55% and isopropanol (IPA) at a final concentration (w/w) of between about 25% to about 60% in the pharmaceutical composition; wherein the DGME is present at a final concentration (w/w) of about 15% and about 35%; and wherein the constituted formulation was formed by combining: a) a first solution of a green porphyrin present in an initial concentration (w/w) of between about 0.5% and 1.5% dissolved in benzyl alcohol; and b) a second solution of a diluent component comprising DGME present at an initial concentration (w/w) of between about 15% and about 40%, benzyl alcohol present at an initial concentration (w/w) of between about 0% and about 30%, and about 40% and isopropanol (IPA) present at an initial concentration (w/w) of between about 30% and about 70% in the pharmaceutical composition; wherein the concentration of the photosensitizer in the constituted solution is supersaturated at ambient temperature. In one embodiment, the constituted pharmaceutical composition is physically stable for at least 4 hours. In another embodiment, the diluent component optionally additionally comprises oleyl alcohol present at an initial concentration (w/w) of between 4.0% and 6.0%, menthol present at an initial concentration (w/w) of between 2.5% and 3.0%, methyl salicylate present at a final concentration (w/w) of between 0.5% and 1.5%, and polysorbate 80 present at a final concentration (w/w) of between 0.25% and 0.60%. In a further embodiment, the first solution of a green porphyrin comprises lemuteporfin present in an initial concentration (w/w) of about 1.00% in benzyl alcohol and the diluent component comprises DGME present at an initial concentration (w/w) of about 35.6%, IPA present at an initial concentration (w/w) of about 54.39%, oleyl alcohol present at an initial concentration (w/w) of about 5.56%, menthol present at an initial concentration of about 2.78%, methyl salicylate present at an initial concentration of about 1.11%, and polysorbate 80 present at an initial concentration of about 0.56%. In another embodiment, the first solution of a green porphyrin comprises lemuteporfin present in an initial concentration (w/w) of about 1.00% and benzyl alcohol at a concentration (w/w) of about 99.00% and the diluent component comprises DGME present at an initial concentration (w/w) of about 24.30%, benzyl alcohol present at an initial concentration (w/w) of 28.55%, and IPA present at an initial concentration (w/w) of about 47.15%. In a further embodiment, the first solution of a green porphyrin comprises lemuteporfin present in an initial concentration (w/w) of about 0.60% and benzyl alcohol at a concentration (w/w) of about 99.40%; and the diluent component comprises DGME present at an initial concentration (w/w) of about 34.00%, and IPA present at an initial concentration (w/w) of about 66.00%. In another embodiment, the green porphyrin is lemuteporfin.

In another aspect, the present invention provides a method of using the described pharmaceutical composition in treating acne in a subject in need thereof, comprising applying a therapeutically effective amount of said composition to an affected area of the subject's skin having acne lesions, allowing sufficient time for at least some of the green porphyrin to localize in the sebaceous glands of the affected area, and exposing the skin of the subject to light energy at a wavelength capable of activating the green porphyrin.

In another aspect, the present invention provides a method of using the described pharmaceutical composition in reducing the sebum excretion rate of sebaceous glands in the skin of a subject having an affected area of oily skin, comprising applying a therapeutically effective amount of said pharmaceutical composition to the affected area, allowing sufficient time for at least some of the composition to localize in the sebaceous glands, and exposing the skin of the subject to light energy at a wavelength capable of activating the photosensitizer.

In a further aspect, the present invention provides a method of preparing the described pharmaceutical composition, comprising mixing a first vial having a photosensitizing component comprising a green porphyrin and benzyl alcohol and a second vial having a diluent component comprising diethylene glycol monoethyl ether (DGME) and isopropanol (IPA) and optionally benzyl alcohol, wherein said pharmaceutical composition has a final concentration (w/w) of between about 0.1% to about 0.4% of said green porphyrin, of between about 5% and about 55% of said benzyl alcohol, of between about 7% and about 25% of said DGME, and of between about 25% and about 60% of said IPA. In one embodiment, the method comprises mixing a first vial comprising a solution of a green porphyrin comprising lemuteporfin present in an initial concentration (w/w) of about 1.00% in benzyl alcohol and a second vial comprising a solution of DGME present at an initial concentration (w/w) of about 35.6%, IPA present at an initial concentration (w/w) of about 54.39%, oleyl alcohol present at an initial concentration (w/w) of about 5.56%, menthol present at an initial concentration of about 2.78%, methyl salicylate present at an initial concentration of about 1.11%, and polysorbate 80 present at an initial concentration of about 0.56%. In another embodiment, the method comprises mixing a first vial comprising a solution of a green porphyrin comprising lemuteporfin present in an initial concentration (w/w) of about 1.00% and benzyl alcohol at a concentration (w/w) of about 99.00%; and a second vial comprises a solution of DGME present at an initial concentration (w/w) of about 24.30%, benzyl alcohol present at an initial concentration (w/w) of 28.55%, and IPA present at an initial concentration (w/w) of about 47.15%. In a further embodiment, the method comprises mixing a first vial comprising a solution of a green porphyrin comprising lemuteporfin present in an initial concentration (w/w) of about 0.60% and benzyl alcohol at a concentration (w/w) of about 99.40%; and a second vial comprises a solution of DGME present at an initial concentration (w/w) of about 34.00%, and IPA present at an initial concentration (w/w) of about 66.00%. In another embodiment, the green porphyrin is lemuteporfin.

In another aspect, the present invention provides a method for reducing the sebum excretion rate of sebaceous glands in the skin of a subject having an area of oily skin, comprising applying a therapeutically effective amount of the described pharmaceutical composition to the affected area on the skin of the subject, allowing sufficient time for at least some of the photosensitizer to localize in the sebaceous glands; and exposing the skin of the subject to light energy at a wavelength capable of activating the photosensitizer. In one embodiment, the photosensitizer is a green porphyrin. In a further embodiment, the green porphyrin is lemuteporfin. In another embodiment, the affected area of the subject is pre-treated with dry heat before the composition is applied. In a further embodiment, the time allowed for the photosensitizer to localize is 1 to 2 hours. In another embodiment, the light energy exposure is in the range of 37.5 to 300 J/cm².

In a further aspect, the present invention provides a method of treating acne in a subject in need thereof comprising applying a therapeutically effective amount of the photosensitizer composition described above to an affected area of the subject's skin having acne lesions, allowing sufficient time for at least some of the photosensitizer to localize in the sebaceous glands of the affected area, and exposing the skin of the subject to light energy at a wavelength capable of activating the photosensitizer. In one embodiment, the subject has inflammatory acne lesions, non-inflammatory acne lesions or both inflammatory and non-inflammatory lesions. In another embodiment, the photosensitizer is a green porphyrin. In a further embodiment, the green porphyrin is lemuteporfin. In a further embodiment, the affected area of the subject is pre-treated with dry heat before the composition is applied. In another embodiment, the time allowed for the photosensitizer to localize is 1 to 2 hours. In a further embodiment, the light energy exposure is in the range of 37.5 to 300 J/cm².

The present disclosure also includes and provides a kit comprising a first container containing a photosensitizing component comprising a photosensitizer, and a second container containing an excipient component that is miscible with the solvents in the first container, and a set of instructions for combining the contents of the two containers, topically applying the combined contents to the skin of a subject, and performing PDT for the treatment of one or more skin disorders.

In certain embodiments according to the present disclosure, the photosensitizers include green porphyrins such as lemuteporfin and verteporfin.

### BRIEF DESCRIPTION OF THE DRAWINGS

FIG. 1 is a graph showing the effect on mouse sebaceous glands of an embodiment according to the present disclosure of PDT with various solution formulations of lemuteporfin (LT-G-001-LT-G-005 shown in Table 4; with and without cellulose gelling agents) and an ointment formulation (LTO-TG1) with red light doses of 50 or 100 J/cm² delivered at an intensity of 50 mW/cm². Flank skin samples obtained 72 hours post-PDT are assessed for numbers of Oil Red O-positive PSU (□) which indicates the presence of sebaceous glands, and the total number of hair follicles (▪) counted within each 4× microscopic field. Mean values with standard deviations for 5 mice per treatment group are presented.

FIG. 2 is a graph comparing the effect of PDT with lemuteporfin in a lemuteporfin topical solution (LTS; LT-G-002-type) in comparison to a lemuteporfin topical ointment (LTO; TG1-type) combined with red light doses of 20, 50 or 100 J/cm² at an intensity of 50 mW/cm². Control mice receive an application of matched formulation that did not contain lemuteporfin and then are exposed to the highest red light dose. Sections prepared from flank skin samples are obtained 72 hours post-PDT and are assessed for Oil Red O-positive PSU (□) and total hair follicles (▪) within each 4× microscopic field. Mean values with standard deviations for 5 mice per treatment group are presented.

FIG. 3 is a bar graph showing lemuteporfin fluorescence intensity measurement in hair follicles and sebaceous glands in human cadaver skin samples comparing a lemuteporfin topical ointment (LTO) at 1 hour and 8 hours after application of lemuteporfin-containing formulation and a lemuteporfin topical solution (F-C) after 1 hour skin contact according to an aspect of the present disclosure.

FIG. 4 shows representative images of upper back sebaceous glands containing lemuteporfin-related fluorescence for different subjects in Cohort 2 from Example 9 following skin preparation and topical application of LTS at 0.1% according to certain aspects of the present disclosure. The upper four fluorescence images are from sites pretreated with infrared red (IR) heat followed by LTS at 0.1%. The lower four images are from skin sites dosed with LTS at 0.1% for 60 minutes without any skin pretreatment.

FIG. 5 is a plot showing the diffusion lemuteporfin from Batch C, formulation TK1 (control) and Batch U, formulation F21 into artificial sebum over time in a closed system at 32.5° C.

FIG. 6 is a plot showing the diffusion lemuteporfin from Batch C, formulation TK1 (control) and Batch U, formulation F21 into artificial sebum over time in an open system at 32.5° C.

FIG. 7 is a plot showing the diffusion lemuteporfin from Batch C, formulation TK1 (control) and Batch U, formulation F21 into artificial sebum over time in a closed system at 35.0° C.

### DETAILED DESCRIPTION OF THE INVENTION

### Overview

The present disclosure provides for and includes pharmaceutical compositions comprising photosensitizers, and methods of using the formulated photosensitizers to perform photodynamic therapy (PDT) for the treatment of dermatological disorders such as acne vulgaris and other hyperactive sebaceous gland disorders.

In order to perform PDT for sebaceous gland disorders, it is necessary to deliver photosensitizer into sebaceous glands. We observed that a previously known ointment formulation of the photosensitizer drug lemuteporfin, similar to that described in WO 03/039597, when applied to the skin of mice, was effective in localizing the photosensitizer to the sebaceous glands of this species. However, the same formulation was not generally as effective in localizing the drug to the sebaceous glands of humans. Therefore, we sought improved formulations that, when applied to human skin, would be capable of delivering an increased amount of a photosensitizer drug to sebaceous glands, preferably in a decreased amount of time.

Unexpectedly, we found that formulations of photosensitizer in the form of a liquid solution, without the addition of substantial amounts of viscosity modifying agents, such as thickeners, gelling agents, waxes, etc., are more effective than formulations such as gels, ointments, lotions, creams, etc. We discovered that addition of gelling agents such as hydroxy-propyl cellulose or ethyl cellulose in substantial amounts actually rendered the formulations relatively less capable of delivering photosensitizer to the sebaceous gland of either mice or humans. Such viscosity-modifying agents are frequently used in conventional topical therapies, and are considered generally useful in stabilizing supersaturated solution because they act as anti-nucleating agents.

We found that the most effective solution formulations that we developed contained concentrations of photosensitizer drugs that are approaching, and preferably exceeding, the solubility of the drug in the formulations. Very surprisingly, solutions of green porphyrins such as lemuteporfin formulated above their solubility (supersaturated solutions) are stable upon storage for up to 4 hours, 8 hours, 12 hours, 24 hours, 32 hours, 48 hours, or more, even without the addition of anti-nucleating or gelling agents (for example polymers such as hydroxyl alkyl celluloses like hydroxypropyl methylcellulose (HPMC), hydroxypropyl cellulose (HPC), polyvinylpyrrolidone (PVP) and polyacrylic acid) that are typically used in the art to prevent precipitate from forming in a supersaturated solution. In certain embodiments, stable supersaturated solutions of green porphyrins such as lemuteporfin are provided, having a concentration of photosensitizer dissolved in solution at amounts of greater than about 150%, greater than about 200%, greater than about 250%, greater than about 260%, greater than about 275%, greater than about 280%, greater than about 300%, greater than about 325%, greater than about 350%, greater than about 375%, greater than about 400% greater than about 425%, etc. of equilibrium solubility at ambient temperature.

For example, the solubility of lemuteporfin in certain pharmaceutical formulations of the present disclosure described herein ranges from about 0.025% to about 0.037% (depending on whether surfactants may be added and depending on what solvents are present and in what ratio). To achieve a concentration in the final formulation in the range of 0.05 to 0.5%, (which we have determined to be in an effective concentration range for performing PDT), a supersaturated solution is desirable. The unexpected stability of such supersaturated solutions for periods of time exceeding 4 hours was an important discovery in view of our observation (herein below) that the presence of polymers typically used in the art as anti-nucleating agents to prevent the precipitation of active ingredients from supersaturated solutions interfered with the localization of lemuteporfin to sebaceous glands. Hence the formulations described herein allow a relatively high concentration of lemuteporfin to be used, while maintaining lemuteporfin in solution for an amount of time that is therapeutically and commercially useful.

### Photosensitizer Formulations

As used herein the term “excipient” means the component(s) of a drug product other than the active pharmaceutical ingredient (API), including pharmaceutically acceptable diluents, vehicles, carriers, solvents, preservatives, antioxidants, viscosity modifying agents or combinations thereof. Unless otherwise indicated, concentrations are disclosed on a w/w % basis.

As used herein, the term “solvent” means a pharmaceutically acceptable liquid solvent capable of dissolving a photosensitizer.

As used herein, the term “supersaturated” or “supersaturated solution” means, with respect to a photosensitizer, that the amount of photosensitizer dissolved in a solution exceeds the equilibrium solubility at a given temperature, usually ambient temperature or 20° C. unless otherwise indicated.

As used herein, the term “solubility” or “saturation solubility” means, with respect to a photosensitizer, the amount of the photosensitizer that can be dissolved in a given solvent at a given temperature at equilibrium, usually ambient temperature or 20° C. unless otherwise indicated.

In one aspect, the present disclosure includes and provides a pharmaceutical composition useful for localizing a photosensitizer to a sebaceous gland, comprising a photosensitizer component and an excipient component in a solution, wherein the concentration of the photosensitizer in the solution is supersaturating, and wherein the photosensitizer does not precipitate out of solution to a pharmaceutically unacceptable degree after the solution is made. Without being bound by any particular theory, it is thought that such supersaturated solutions are highly effective delivery systems for large molecules like lemuteporfin because the thermodynamic activity of the photosensitizer in the vehicle being at its highest and the resulting high concentration gradient being further increased by the evaporation of some of the volatile formulation components, lemuteporfin effectively partitions into the sebum, the waxy/oily mixture secreted by sebaceous glands, and the living cells (sebocytes) which comprise the PSU and sebaceous glands.

The present disclosure further includes and provides a pharmaceutical composition comprising a solubilized photosensitizer and optionally, other excipients, wherein the concentration of photosensitizer in the composition exceeds the saturation solubility of the photosensitizer in the composition.

The present disclosure also includes and provides a composition useful for topical delivery of a photosensitizer comprising a photosensitizer, one or more solvents and optionally one or more pharmaceutically acceptable excipients, wherein the composition has a viscosity of less than 50 centipoise (cps) at 20° C. Such a composition contains no (or very low amounts of) viscosity-modifying agents, and may be supersaturated or not.

The photosensitizer component in the compositions may be present at concentrations ranging from about 0.001% to about 5% (w/w) depending on the type of photosensitizer chosen, its potency and its solubility. Typically, the photosensitizer component is present at concentrations ranging from about 0.01% to about 1.0%. For green porphyrins, such as lemuteporfin, concentrations may range from 0.025% to about 0.5%. In an aspect the concentration may be 0.025% or 0.05%. In another aspect the concentration may be 0.075% or 0.1%. In other aspects the concentration may be 0.125% or 0.15%. In a further aspect the concentration may be 0.175% or 0.2%. In an aspect, the concentration may be 0.225% or 0.25%. In yet another aspect, the concentration may be 0.3% or 0.355%. In certain aspects, the concentration may be 0.375%, 0.4% or 0.5%. In certain aspects according to the present disclosure, the concentration of a green porphyrin may be in the range of 0.05% to 0.4%. In certain aspects according to the present disclosure, the concentration of a green porphyrin may be in the range of about 0.3% to about 0.4%. In other aspects, the concentration of a green porphyrin may be in the range 0.35% to 0.45%. In another aspect, the concentration of lemuteporfin may be in the range of about 0.1% to about 0.3%.

The excipient component in the compositions typically includes one or more solvents for the photosensitizer, such as benzyl alcohol (a solvent for green porphyrins such as lemuteporfin), DGME (diethylene glycol monoethyl ether), isopropyl alcohol, or combinations thereof. In some embodiments, benzyl alcohol may be present in concentrations (w/w) ranging from about 1% to about 50% or more, about 1% to about 40%, about 1% to about 30%, about 1% to about 20%, about 5% to about 50%, or about 20% to about 50%, such as 5%, 6%, 7%, 8%, 9%, 10%, 11%, 12%, 13%, 14%, 15%, 20%, 25%, 30%, 35%, 40%, 45%, 50% etc. In some embodiments, the benzyl alcohol may be present in an amount greater than about 20%, about 20% to about 50%, about 25% to about 50%, about 30% to about 50%, about 40% to about 50%, about 45% to about 50%, etc. In one embodiment, benzyl alcohol is present at about 10%, about 39.8%, about 46.9%, or about 49.6%. In other aspects according the present disclosure, the benzyl alcohol solvent may be from 35 to 50% (w/w). In other aspects, the benzyl alcohol solvent may be from 40% to 50% or 45% to 50%.

In some embodiments DGME may be present in the diluent component in concentrations (w/w) ranging from about 5% to about 50% or more, from about 10% to about 40%, or from about 15% to about 35% such as 15%, 16%, 17%, 18%, 19%, 20%, 21%, 22%, 23%, 24%, 25%, 26%, 27%, 28%, 29%, 30%, 31%, 32%, 33%, 34%, 35% and 36%. In one embodiment, DGME is present at about 17.5%, about 16.7%, about 20%, or about 32%. In an aspect, DGME may be present in the excipient component at a concentration of between about 15% and about 20%. In an aspect, DGME may be present in the diluent component at a concentration of between about 17.5% and about 22.5%. In an aspect, DGME may be present in the excipient component at a concentration of between about 16.7% and about 22.5%. In an aspect, DGME may be present in the diluent component at a concentration of between about 17.5% and about 32%. In an aspect, DGME may be present in the excipient component at a concentration of between about 16.7% and about 20%.

In some embodiments, isopropyl alcohol may be present in the diluent component in concentrations (w/w) ranging from about 30% to about 85% or more. In other embodiments, isopropyl alcohol may be present in the excipient component in concentrations (w/w) ranging from about 40% to about 70%. In yet another aspect, isopropyl alcohol may be present in the diluent component in concentrations (w/w) ranging from about 50% to about 60%. In yet another aspect, isopropyl alcohol may be present in the excipient component in concentrations (w/w) ranging from about 30% to about 40%. In some embodiments, isopropyl alcohol is present at 31% or 32%. In some embodiments, isopropyl alcohol is present at 33% or 34%. In some embodiments, isopropyl alcohol is present at 35% or 36%. In some embodiments, isopropyl alcohol is present at 37% or 38%. In yet other embodiments, isopropyl alcohol is present at 39% or 40%. In some embodiments, isopropyl alcohol is present at 41%, 42%, 43%, 44%, 45%, 46%, 47%, 48%, 49%, 50%, 51%, 52%, 53%, 54%, 55%, 56%, 57%, 58%, 59%, or 60%. In one embodiment, isopropyl alcohol is present at about 33.3%, about 35.2%, about 39.8%, or about 49%.

In some embodiments, acetone may be present in the excipient component at concentrations (w/w) ranging from 0% to about 10% or more, or about 2% to about 10%. In some embodiments, oleyl alcohol may be present in the diluent component at concentrations (w/w) ranging from 0% to about 6% or more, or about 2% to 5%. In one embodiment, oleyl alcohol is present at 5%. In some embodiments, polysorbate 80 may be present in the diluent component in concentrations ranging from 0% to about 1% or more, or about 0.25% to about 0.75%. In one embodiment, polysorbate 80 is present at 0.5%. In some embodiments, methyl salicylate is present in the diluent component in concentrations (w/w) ranging from 0% to about 2% or more, about 0.5% to about 1.5% or about 0.075% to about 1.25%. In one embodiment, methyl salicylate is present at about 1.0%. In some embodiments, menthol is present in the excipient component in concentrations (w/w) ranging from 0% to about 6% or more, about 1% to about 5% or about 2% to about 3%. In one embodiment, menthol is present at 2.5%.

Other solvents and excipients for photosensitizers may also include DMSO (dimethylsulfoxide), polyethylene glycol (PEG), PEG derivatives, glycol ethers, propylene glycol, polysorbates (e.g., Tween®), fatty alcohols, aromatic alcohols, glycerols, oils, surfactants, glucosides, thiethylene glycol, tetraethylene glycol, pentaethylene glycol, hexaethylene glycol, septathylene glycol, octaehtylene glycol, propylene glycol, propylene glycol mono- and di-esters of fats and fatty acids (e.g., propylene glycol monocaprylate, propylene glychol monolaurate), glycerol, mineral oil, lanolin, petrolatum or other petroleum products suitable for application to the skin, macrogols, macrogolglycerides or polyethylene glycol glycerides and fatty esters (e.g., stearoyl macrogolglycerides, oleoyl macrogolglycerides, lauroyl macrogolglycerides, linoleoyl macrogolglycerides), ethoxylated castor oil (e.g., Cremophor, a polyoxyl hydrogenated castor oil), C₆-C₃₀ triglycerides, natural oils, glucosides (e.g., cetearl glucosides and surfactants).

In an embodiment according the present disclosure, a pharmaceutical composition useful for localizing a photosensitizer to a sebaceous gland may comprise a photosensitizing component having a photosensitizer that is a green porphyrin, including lemuteporfin, present at a concentration (w/w) of between 0.09% to 0.11% and a solvent comprising benzyl alcohol at a concentration (w/w) of between about 31.9% and about 32.1%, and a diluent component comprising diethylene glycol monoethyl ether (DGME) present at a concentration (w/w) of between about 4.95% and about 5.05% and isopropanol (IPA) present at a concentration (w/w) of between about 53.8% and about 54%. In another embodiment, the photosensitizer, including lemuteporfin, may be present at a concentration of about 0.1% and benzyl alcohol at a concentration of 32%, and a diluent component having DGME at 5% and IPA at 53.9%.

In an embodiment according the present disclosure, a pharmaceutical composition useful for localizing a photosensitizer to a sebaceous gland may comprise a photosensitizing component having a photosensitizer that is a green porphyrin, including lemuteporfin, present at a concentration (w/w) of between 0.065% to 0.085% and a solvent comprising benzyl alcohol at a concentration (w/w) of between about 31.9% and about 32.1%, and a diluent component comprising diethylene glycol monoethyl ether (DGME) present at a concentration (w/w) of between about 4.95% and about 5.05% and isopropanol (IPA) present at a concentration (w/w) of between about 53.825% and about 54.025%. In another embodiment, the photosensitizer, including lemuteporfin, may be present at a concentration of about 0.075% and benzyl alcohol at a concentration of 32%, and a diluent component having DGME at 5% and IPA at 53.925%.

In an embodiment according the present disclosure, a pharmaceutical composition useful for localizing a photosensitizer to a sebaceous gland may comprise a photosensitizing component having a photosensitizer that is a green porphyrin, including lemuteporfin, present at a concentration (w/w) of between 0.09% to 0.11% and a solvent comprising benzyl alcohol at a concentration (w/w) of between about 9.9% and about 10.1%, and a diluent component comprising diethylene glycol monoethyl ether (DGME) present at a concentration (w/w) of between about 31.95% and about 32.05% and isopropanol (IPA) present at a concentration (w/w) of between about 48.8% and about 49%. In another embodiment, the photosensitizer, including lemuteporfin, may be present at a concentration of about 0.1% and benzyl alcohol at a concentration of 10%, and a diluent component having DGME at 32% and IPA at 48.9%.

In an embodiment according the present disclosure, a pharmaceutical composition useful for localizing a photosensitizer to a sebaceous gland may comprise a photosensitizing component having a photosensitizer that is a green porphyrin, including lemuteporfin, present at a concentration (w/w) of between 0.065% to 0.085% and a solvent comprising benzyl alcohol at a concentration (w/w) of between about 9.9% and about 10.1%, and a diluent component comprising diethylene glycol monoethyl ether (DGME) present at a concentration (w/w) of between about 31.95% and about 32.05% and isopropanol (IPA) present at a concentration (w/w) of between about 48.825% and about 49.025%. In another embodiment, the photosensitizer, including lemuteporfin, may be present at a concentration of about 0.075% and benzyl alcohol at a concentration of 10%, and a diluent component having DGME at 32% and IPA at 48.925%.

In an embodiment according the present disclosure, a pharmaceutical composition useful for localizing a photosensitizer to a sebaceous gland may comprise a photosensitizing component having a photosensitizer that is a green porphyrin, including lemuteporfin, present at a concentration (w/w) of between 0.04% to 0.06% and a solvent comprising benzyl alcohol at a concentration (w/w) of between about 9.9% and about 10.1%, and a diluent component comprising diethylene glycol monoethyl ether (DGME) present at a concentration (w/w) of between about 31.95% and about 32.05% and isopropanol (IPA) present at a concentration (w/w) of between about 48.85% and about 49.05%. In another embodiment, the photosensitizer, including lemuteporfin, may be present at a concentration of about 0.05% and benzyl alcohol at a concentration of 10%, and a diluent component having DGME at 32% and IPA at 48.95%.

In an embodiment according the present disclosure, a pharmaceutical composition useful for localizing a photosensitizer to a sebaceous gland may comprise a photosensitizing component having a photosensitizer that is a green porphyrin, including lemuteporfin, present at a concentration (w/w) of between 0.11% to 0.13% and a solvent comprising benzyl alcohol at a concentration (w/w) of between about 11.8% and about 12%, and a diluent component comprising diethylene glycol monoethyl ether (DGME) present at a concentration (w/w) of between about 22.45% and about 22.55% and isopropanol (IPA) present at a concentration (w/w) of between about 57.9% and about 58.1%. In another embodiment, the photosensitizer, including lemuteporfin, may be present at a concentration of about 0.12% and benzyl alcohol at a concentration of 11.9%, and a diluent component having DGME at 22.5% and IPA at 58%.

In an embodiment according the present disclosure, a pharmaceutical composition useful for localizing a photosensitizer to a sebaceous gland may comprise a photosensitizing component having a photosensitizer that is a green porphyrin, including lemuteporfin, present at a concentration (w/w) of between 0.11% to 0.13% and a solvent comprising benzyl alcohol at a concentration (w/w) of between about 11.9% and about 12.1%, and a diluent component comprising diethylene glycol monoethyl ether (DGME) present at a concentration (w/w) of between about 22.75% and about 22.85% and isopropanol (IPA) present at a concentration (w/w) of between about 58.5% and about 58.7%. In another embodiment, the photosensitizer, including lemuteporfin, may be present at a concentration of about 0.12% and benzyl alcohol at a concentration of 12%, and a diluent component having DGME at 22.8% and IPA at 58.6%.

In an embodiment according the present disclosure, a pharmaceutical composition useful for localizing a photosensitizer to a sebaceous gland may comprise a photosensitizing component having a photosensitizer that is a green porphyrin, including lemuteporfin, present at a concentration (w/w) of between 0.12% to 0.14% and a solvent comprising benzyl alcohol at a concentration (w/w) of between about 12.7% and about 12.9%, and a diluent component comprising diethylene glycol monoethyl ether (DGME) present at a concentration (w/w) of between about 24.35% and about 24.45% and isopropanol (IPA) present at a concentration (w/w) of between about 62.6% and about 62.8%. In another embodiment, the photosensitizer, including lemuteporfin, may be present at a concentration of about 0.13% and benzyl alcohol at a concentration of 12.8%, and a diluent component having DGME at 24.4% and IPA at 62.7%.

In an embodiment according the present disclosure, a pharmaceutical composition useful for localizing a photosensitizer to a sebaceous gland may comprise a photosensitizing component having a photosensitizer that is a green porphyrin, including lemuteporfin, present at a concentration (w/w) of between 0.33% to 0.35% and a solvent comprising benzyl alcohol at a concentration (w/w) of between about 48.66% and about 48.86%, and a diluent component comprising diethylene glycol monoethyl ether (DGME) present at a concentration (w/w) of between about 16.89% and about 16.99% and isopropanol (IPA) present at a concentration (w/w) of between about 33.86% and about 34.06%. In another embodiment, the photosensitizer, including lemuteporfin, may be present at a concentration of about 0.34% and benzyl alcohol at a concentration of 48.76%, and a diluent component having DGME at 16.94% and IPA at 33.96%.

In an embodiment according the present disclosure, a pharmaceutical composition useful for localizing a photosensitizer to a sebaceous gland may comprise a photosensitizing component having a photosensitizer that is a green porphyrin, including lemuteporfin, present at a concentration (w/w) of between 0.39% to 0.41% and a solvent comprising benzyl alcohol at a concentration (w/w) of between about 39.7% and about 39.9%, and a diluent component comprising diethylene glycol monoethyl ether (DGME) present at a concentration (w/w) of between about 19.95% and about 20.05% and isopropanol (IPA) present at a concentration (w/w) of between about 39.7% and about 39.9%. In another embodiment, the photosensitizer, including lemuteporfin, may be present at a concentration of about 0.4% and benzyl alcohol at a concentration of 39.8%, and a diluent component having DGME at 20% and IPA at 39.8%.

In an embodiment according the present disclosure, a pharmaceutical composition useful for localizing a photosensitizer to a sebaceous gland may comprise a photosensitizing component having a photosensitizer that is a green porphyrin, including lemuteporfin, present at a concentration (w/w) of between 0.49% to 0.51% and a solvent comprising benzyl alcohol at a concentration (w/w) of between about 49.7% and about 49.9%, and a diluent component comprising diethylene glycol monoethyl ether (DGME) present at a concentration (w/w) of between about 24.85% and about 24.95% and isopropanol (IPA) present at a concentration (w/w) of between about 24.8% and about 25%. In another embodiment, the photosensitizer, including lemuteporfin, may be present at a concentration of about 0.5% and benzyl alcohol at a concentration of 49.8%, and a diluent component having DGME at 24.9% and IPA at 24.9%.

In an embodiment according the present disclosure, a pharmaceutical composition useful for localizing a photosensitizer to a sebaceous gland may comprise a photosensitizing component having a photosensitizer that is a green porphyrin, including lemuteporfin, present at a concentration (w/w) of between 0.39% to 0.41% and a solvent comprising benzyl alcohol at a concentration (w/w) of between about 39% and about 39.2%, and a diluent component comprising diethylene glycol monoethyl ether (DGME) present at a concentration (w/w) of between about 19.45% and about 19.55% and isopropanol (IPA) present at a concentration (w/w) of between about 31.2% and about 31.4%. In another embodiment, the photosensitizer, including lemuteporfin, may be present at a concentration of about 0.4% and benzyl alcohol at a concentration of 39.1%, and a diluent component having DGME at 19.5% and IPA at 31.3%.

In an embodiment according the present disclosure, a pharmaceutical composition useful for localizing a photosensitizer to a sebaceous gland may comprise a photosensitizing component having a photosensitizer that is a green porphyrin, including lemuteporfin, present at a concentration (w/w) of between 0.345% to 0.365% and a solvent comprising benzyl alcohol at a concentration (w/w) of between about 46.85% and about 47.05%, and a diluent component comprising diethylene glycol monoethyl ether (DGME) present at a concentration (w/w) of between about 17.49% and about 17.59% and isopropanol (IPA) present at a concentration (w/w) of between about 35.06% and about 35.26%. In another embodiment, the photosensitizer, including lemuteporfin, may be present at a concentration of about 0.355% and benzyl alcohol at a concentration of 46.95%, and a diluent component having DGME at 17.54% and IPA at 35.16%.

In an embodiment according the present disclosure, a pharmaceutical composition useful for localizing a photosensitizer to a sebaceous gland may comprise a photosensitizing component having a photosensitizer that is a green porphyrin, including lemuteporfin, present at a concentration (w/w) of between 0.365% to 0.385% and a solvent comprising benzyl alcohol at a concentration (w/w) of between about 49.53% and about 49.73%, and a diluent component comprising diethylene glycol monoethyl ether (DGME) present at a concentration (w/w) of between about 16.62% and about 16.72% and isopropanol (IPA) present at a concentration (w/w) of between about 33.23% and about 33.43%. In another embodiment, the photosensitizer, including lemuteporfin, may be present at a concentration of about 0.375% and benzyl alcohol at a concentration of 49.63%, and a diluent component having DGME at 16.67% and IPA at 33.33%.

In an embodiment according the present disclosure, a pharmaceutical composition useful for localizing a photosensitizer to a sebaceous gland may comprise a photosensitizing component having a photosensitizer that is a green porphyrin, including lemuteporfin, present at a concentration (w/w) of between 0.344% to 0.364% and a solvent comprising benzyl alcohol at a concentration (w/w) of between about 46.788% and about 46.988%, and a diluent component comprising diethylene glycol monoethyl ether (DGME) present at a concentration (w/w) of between about 17.667% and about 17.767% and isopropanol (IPA) present at a concentration (w/w) of between about 34.94% and about 35.14%. In another embodiment, the photosensitizer, including lemuteporfin, may be present at a concentration of about 0.354% and benzyl alcohol at a concentration of 46.888%, and a diluent component having DGME at 17.717% and IPA at 35.04%.

An additional embodiment of a combined two-component solution comprises a concentration (w/w) of 0.10% lemuteporfin, benzyl alcohol at a concentration of 10.0%, isopropyl alcohol at a concentration of 48.9%, DGME at a concentration of 32.0%, oleyl alcohol at a concentration of 5.0%, menthol at a concentration of 2.5%, methyl salicylate at a concentration at a concentration of 1.0%, and polysorbate 80 at a concentration of 0.50%. A further embodiment comprises a final concentration (w/w) of 0.30% lemuteporfin, benzyl alcohol at a concentration of 49.7%, isopropyl alcohol at 33.0%, and DGME at a concentration of 17.0%. <NOTE: Re Delphine's comment, the previous sentence is meant to describe LTS, 0.3% constituted solution from Table 38. Even though it may be the same as TK1 and described elsewhere in the spec, there's no harm in repeating it here. It was difficult for me to understand exactly how some of the old formulations related to the new formulations and thus I erred on the side of caution and added all of the new information even if it was repetitive. We want to be able to draw a patent examiner's attention to easily understandable disclosures, even if the application is very repetitive.>

In an embodiment according the present disclosure, a pharmaceutical composition useful for localizing a photosensitizer to a sebaceous gland may comprise a diluent component comprising isopropyl alcohol present at a concentration (w/w) of 54.39%, DGME present at a concentration of 35.60%, oleyl alcohol present at a concentration of 5.56%, menthol present at a concentration of 2.78%, methyl salicylate present at a concentration of 1.11%, and polysorbate 80 present at a concentration of 0.56%. This diluent component may be mixed with a lemuteporfin photosensitizing component, comprising lemuteporfin at a concentration of 1.00% and benzyl alcohol at a concentration of 99.00% to yield a combined solution comprising 0.1% lemuteporfin.

In a further embodiment, the pharmaceutical composition may comprise a diluent component comprising benzyl alcohol present at a concentration (w/w) of 28.55%, isopropyl alcohol present at a concentration of 47.15%, and DGME present at a concentration of 24.30%. This diluent component may be mixed with a lemuteporfin photosensitizing component, comprising lemuteporfin at a concentration of 1.00% and benzyl alcohol at a concentration of 99.00% to yield a combined solution comprising 0.3% lemuteporfin.

In a further embodiment, the pharmaceutical composition may comprise a diluent component comprising isopropyl alcohol present at a concentration (w/w) of 66.00%, and DGME present at a concentration of 34.00%. This diluent component may be mixed with a lemuteporfin photosensitizing component, comprising lemuteporfin at a concentration of 0.60% and benzyl alcohol at a concentration of 99.40% to yield a combined solution comprising 0.3% lemuteporfin.

In a further embodiment, the pharmaceutical composition may comprise a diluent component comprising isopropyl alcohol present at a concentration (w/w) of 54.39%, DGME present at a concentration of 35.60%, oleyl alcohol present at a concentration of 5.56%, menthol present at a concentration of 27.8%, methyl salicylate present at a concentration of 1.11%, and polysorbate 80 present at a concentration of 0.56%. This diluent component may be mixed with a lemuteporfin photosensitizing component, comprising lemuteporfin at a concentration of 1.00% and benzyl alcohol at a concentration of 99.00% to yield a combined solution comprising 0.1% lemuteporfin.

In some embodiments, the formulation composition need not contain substantial amounts of viscosity enhancing agents such as thickeners, gelling agents, etc. Such formulation compositions have a viscosity of less than 50 centipoise (cps) at 20° C. If needed or desired, the formulation compositions can be thickened by the addition of such viscosity enhancing agents as high MW polyethylene glycols, celluloses (such as hydroxypropyl cellulose or ethyl cellulose) acrylic acid-based polymers (carbopol polymers or carbomers), polymers of acrylic acid crosslinked with allyl sucrose or allylpentaerythrritol (carbopol homepolymers) polymers of acrylic acid modified by long chain (C10-C30) alkyl acrylates and crosslinked with allylpentaerythritol (carbopol copolymers), poloxamers (also known as pluronics; block polymers e.g., Poloxamer 124, 186, 237, 338, 407, etc.), waxes (paraffin, glyceryl monostearate, diethylene glycol monostearate, propylene glycol monostearate, ethylene glycol monostearate, glycol stearate), hard fats (e.g., saturated C8-C18 fatty acid glycerides), xanthum gum, polyvinyl alcohol, solid alcohols, or mixtures thereof. However, as noted above, care must be taken when using viscosity modifying agents to ensure that they are not used in amounts that will interfere with delivery of the photosensitizers to sebaceous glands. In certain embodiments exemplified herein, it is desirable not to add any viscosity modifying agents.

In certain aspects, the formulations may be prepared such that the concentrations of excipients are generally below the maximum levels listed in the U.S. FDA Inactive Ingredient Guide (IIG). By way of example, the Apr. 13, 2013, IIG levels for exemplary inactive ingredients for topical use include, but are not limited to the below in Table 1. However, those of skill in the art understand that such levels are subject to revision.

TABLE 1
Exemplary Inactive Ingredients

| | Topical | |
|---|---|---|
| Ingredient | Formulation Type | Max IIG |
| Benzyl alcohol | gel | 50% |
| Isopropyl alcohol, USP | lotion | 78% |
| (IPA) | | |
| Diethylene glycol monoethyl | gel | 25% |
| ether | | |
| Oleyl alcohol, NF | cream | 10% |
| Menthol, USP | solution | 0.08% |
| Methyl salicylate, NF | gel | 1% |
| Polysorbate 80, NF | lotion | 9.40% |

A supersaturated formulation of photosensitizer can be made in a number of ways. In one embodiment, a photosensitizer is dissolved in a good solvent for the photosensitizer (with or without heating), and then other excipients, in which the photosensitizer is less soluble, are added. In another embodiment, a suspension of photosensitizer and solvent(s), and optionally other excipients, can be heated until an amount of photosensitizer exceeding the solubility in the solvent(s) has been completely dissolved. In another embodiment, a photosensitizer is added below saturation solubility to one or more solvents(s) having one or more volatile components, such as ethanol, water, propanol, isopropanol or other volatile liquids known in the art. The volatile components evaporate to create a supersaturated condition in the less volatile components. For example, a non-saturated photosensitizer formulation for the treatment of acne can be prepared in excipients comprising volatile components. When the photosensitizer formulation is applied to the skin of a subject, some of the volatile components evaporate, creating a supersaturated solution in situ. In yet another embodiment, a supersaturated solution is prepared in excipients with one or more volatile components, and then further supersaturation occurs when the solution is applied to the skin of a subject as the volatile components evaporate.

### Long Term Stability of Formulations

We have found that supersaturated solutions of lemuteporfin are physically stable (i.e., lemuteporfin does not begin to precipitate out of solution) for at least 4 hours, 8 hours, 12 hours, 24 hours, 32 hours, 48 hours, or more. If the desired concentration of the photosensitizer in the formulation composition exceeds the saturation solubility, and long term stability/shelf life (for example, 1-2 years) of the composition is desired, then it may be advantageous to provide a two-component formulation (or a multi-component formulation) where the components may be stored separately, and mixed prior to use. Further, it may be advantageous that the separate component vials are not supersaturated themselves, but provide a supersatured solution when the separate components are mixed together.

Hence in another embodiment, a supersaturated solution is prepared by mixing a solution containing the photosensitizer component with a second solution comprising the excipient component, in which the solubility of the photosensitizer is lower. This aspect of the present disclosure provides a pharmaceutical composition useful for localizing a photosensitizer to a sebaceous gland comprising a photosensitizing component comprising a photosensitizer, and associated therewith but separate therefrom, an excipient component, wherein the photosensitizer is present in an amount sufficient to form, on mixing, a supersaturated solution thereof and wherein the photosensitizer does not precipitate out of solution to a pharmaceutically unacceptable degree for a period of at least 4 hours, 8 hours, 12 hours, 24 hours, 32 hours, 48 hours, or more once the photosensitizing component and the excipient component are mixed. Preferably, the two components are miscible, and thus may be easily combined, for example, by gentle shaking, stirring, or swirling.

In a related aspect, the present disclosure further provides a two-component pharmaceutical composition comprising two liquid phases, wherein at least one of the liquid phases comprises a photosensitizer dissolved therein, the two liquid phases are miscible, and the first liquid phase and the second liquid phase have different solubilities for the photosensitizer, and wherein the concentration of the photosensitizer in each liquid phase is such that, upon combination of the two liquid phases, the total photosensitizer concentration in the liquid mixture is greater than the solubility of the photosensitizer in that liquid mixture, and the resulting liquid mixture is supersaturated with the photosensitizer. In an alternative embodiment, the photosensitizer is provided as a solid phase, rather than as a liquid solution. The photosensitizer solid is dissolved in a solvent prior to, or simultaneously with, mixing of the photosensitizer with the second liquid phase. The solid photosensitizer may be made amorphous or micronized to decrease the time to dissolution.

In some embodiments, the two component formulation comprises a first photosensitizing component comprising lemuteporfin dissolved in benzyl alcohol, with or without DGME. In some embodiments, the two component formulation comprises a second diluent component comprising DGME and/or isopropyl alcohol, and optionally benzyl alcohol. In some embodiments the diluent component additionally comprises oleyl alcohol, menthol, methyl salicylate, or polysorbate 80. The concentrations of the elements of the photosensitizing component and the diluent component are adjusted so that the when the two components are combined, the final concentrations of the elements are in the concentration ranges provided above for lemuteporfin, benzyl alcohol, DGME, isopropanol, oleyl alcohol, menthol, methyl salicylate, and polysorbate 80.

The concentration of photosensitizer in the photosensitizing component may range from the above the saturation solubility in the solvent downward. For a photosensitizing component comprising lemuteporfin dissolved in benzyl alcohol, the apparent solubility following heating is in the range of about 1.0% w/w to 2.5% w/w. By way of example, in one embodiment, a photosensitizing component comprises a 1% w/w solution of lemuteporfin in benzyl alcohol, and prior to use, it is mixed with an diluent component at a ratio of approximately 1 in 10 to give a final concentration of lemuteporfin in the formulation composition of about 0.1% w/w. In another embodiment, the photosensitizing component comprises a 2% solution of lemuteporfin in benzyl alcohol, and prior to use it is mixed with an excipient component at a ratio of approximately 1 in 10 to give a final concentration of lemuteporfin in the formulation composition of about 0.2% w/w. (A similar final product could also be obtained by mixing a photosensitizing component comprising a 1% solution of lemuteporfin with an excipient component at a ratio of 1 in 5.) It can thus be seen that the concentrations in the two components can be adjusted and manipulated to give the desired final concentrations of photosensitizer and excipients in the formulation to be used in PDT. Exemplary methods and compositions for some two-component formulations of the present disclosure are given in the examples below.

In one aspect, the present disclosure provides a method of preparing a pharmaceutical composition comprising the steps of: (a) providing a photosensitizing component comprising a photosensitizer dissolved in a solvent; (b) providing an diluent component miscible with the photosensitizing component; and (c) mixing an amount of the photosensitizing component with an amount of the diluent component to provide a mixed solution, wherein the mixed solution is supersaturated with the photosensitizer. Conveniently, the photosensitizing component and the diluent component may be provided in suitable separate containers, such as glass vials. The photosensitizing component may comprise a green porphyrin, such as lemuteporfin, and the solvent may comprise benzyl alcohol, both in the concentrations described above. The diluent component may comprise diethylene glycol monoethyl ether (DGME) and isopropyl alcohol (IPA), both in the concentrations described above. Optionally, the diluent component may comprise benzyl alcohol, in the concentrations described above.

In order to be useful clinically, the photosensitizer should not precipitate out of the pharmaceutical composition until it is applied to a subject. Preferably, the photosensitizer does not precipitate out of the pharmaceutical composition for at least about 30 seconds, about 1 minute, about 5 minutes, about 15 minutes, about 30 minutes, about 45 minutes or about an hour or more after the photosensitizing component is mixed with the diluent component. In other embodiments, the photosensitizer does not precipitate out of the pharmaceutical composition for at least 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, or at least about 12 hours, or more after the photosensitizing component is mixed with the excipient component. In some embodiments, the photosensitizer does not precipitate out of the pharmaceutical composition for up to at least about 16 hours, at least about 24 hours, at least about 48 hours, at least about 3 days, at least about 5 days, at least about 7 days, at least about 9 days, at least about 11 days, at least about 14 days, at least about 3 weeks, or at least about 4 weeks after the photosensitizing component is mixed with the excipient component. In other embodiments, the photosensitizer may remain dissolved for at least about 2 months, at least about 3 months, at least about 4 months, at least about 5 months or at least about 6 months after the photosensitizing component is mixed with the excipient component. In yet another embodiment, the photosensitizer may remain dissolved for at least about one year or at least about 2 years after the photosensitizing component is mixed with the diluent component.

In order to determine the time at which the photosensitizer may begin to precipitate out of a given pharmaceutical composition of the present disclosure, and hence how long the composition may be kept before use, the compositions may be tested as follows. Samples of the compositions are taken at various time points after combining the photosensitizing component and the diluent component. Half of the samples are filtered to remove any precipitates, for example through a filter of appropriate size, including, but not limited to, a 0.22 μm filter. The filtered solutions are analyzed, for example, using HPLC, for the content or concentration of photosensitizer. If the solution is stable, and no photosensitizer has precipitated out, then the concentration of photosensitizer in the filtered solution should be roughly the same as the concentration of photosensitizer in the unfiltered solution, within experimental error. (This method is carried out in the examples below to demonstrate that the stability of lemuteporfin in a formulation of the present disclosure is at least 4 hours, 8 hours, 12 hours, 24 hours, 32 hours, 48 hours, or more.) If the concentration of photosensitizer in the filtered and unfiltered samples is not roughly the same within experimental error, it may be considered that precipitation to a pharmaceutically unacceptable degree has occurred.

The components of the pharmaceutical composition should be mixed and then applied to the subject within the time period that the photosensitizer remains dissolved in the composition. In some embodiments, the components are combined within about 1 minute to about 24 hours of use. In one embodiment, the components are combined immediately prior to use. In another embodiment, the components are combined within about 30 seconds, about 1 minute, about 5 minutes, about 15 minutes, about 30 minutes, about 45 minutes or about an hour of use. In other embodiments the components are combined within about 1 hour to about 12 hours of use, such as within about 1, about 2, about 3, about 4, about 5, about 6, about 7, about 8, about 9, about 10, about 11, about 12 hours of use. In some embodiments, the components are combined within about 12 to about 24 hours of use, such as within about 12, about 13, about 14, about 15, about 16, about 17, about 18, about 19, about 20, about 21, about 22, about 23 or about 24 hours of use. In some embodiments, the components are combined within about 3 to 4 hours of use.

In another aspect, the present disclosure also includes and provides a kit comprising a first container containing a photosensitizing component comprising a photosensitizer, and one or more containers containing excipient component(s) miscible with the solvents in the first container, and a set of instructions for combining the contents of the containers, topically applying the combined contents to the skin of a subject, and performing PDT for the treatment of one or more skin disorders. In one embodiment the containers are physically separate, for example, two or more vials. In another embodiment, the photosensitizing component and the diluent component(s) are packaged in a single container having two or more chambers that allow the components to be physically segregated from each other initially, and a release system to allow contact between chambers.

### Photosensitizers

As used herein “photosensitizer” or “photosensitizing agent” or “photosensitizing drug” means a chemical compound that absorbs electromagnetic radiation, most commonly in the visible spectrum, and releases it as another form of energy, most commonly as reactive oxygen species and/or as thermal energy. Preferably, the compound is nontoxic to humans or is capable of being formulated in a nontoxic composition. Preferably, the chemical compound produced upon photodegradation is also nontoxic. Hydrophobic and lipophilic photosensitizers tend to be especially useful for use in the compositions and methods of the present disclosure because they may be more effective at partitioning into and diffusing through sebum and localizing in sebaceous glands.

A particularly potent group of photosensitizers is known as green porphyrins, which are described in detail in U.S. Pat. No. 5,171,749, which is incorporated herein by reference in its entirety. The term “green porphyrins” refers to porphyrin derivatives obtained by reacting a porphyrin nucleus with an alkyne in a Diels-Alder type reaction to obtain a mono-hydrobenzoporphyrin. Such resultant macropyrrolic compounds are called benzoporphyrin derivatives (BPDs), which are synthetic chlorin-like porphyrins with various structural analogues and shown in U.S. Pat. No. 5,171,749.

Typically, green porphyrins are selected from a group of tetrapyrrolic porphyrin derivatives obtained by Diels-Alder reactions of acetylene derivatives with protoporphyrin under conditions that promote reaction at only one of the two available conjugated, nonaromatic diene structures present in the protoporphyrin-IX ring systems (rings A and B). Metallated forms of a green porphyrin, in which a metal cation replaces one or two hydrogens in the center of the ring system, may also be used in the practice of the disclosed compositions and methods.

The preparation of green porphyrin compounds useful in this disclosure is described in detail in U.S. Pat. No. 5,095,030, which is incorporated herein in its entirety. Non-limiting examples of green porphyrins include benzoporphyrin diester di-acid (BPD-DA), mono-acid ring A (BPD-MA, also known as verteporfin), mono-acid ring B (BPD-MB), or mixtures thereof. These compounds absorb light of about 692 nm wavelength which has good tissue penetration properties. Particular useful for use herein are the group of green porphyrins known as ethylene glycol esters as set forth in U.S. Pat. No. 5,929,105. The exemplary photosensitizer compound referred to therein as A-EA6 is also known by the generic name lemuteporfin, and has the following chemical structure:

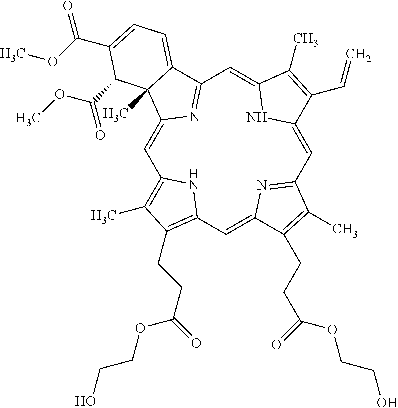 [SMILES: C=Cc1c(C)c2cc3nc(cc4[nH]c(cc5nc(cc1[nH]2)C(C)=C5CCC(=O)OCCO)c(CCC(=O)OCCO)c4C)[C@@]1(C)C3=CC=C(C(=O)OC)[C@H]1C(=O)OC]

Additionally, the photosensitizers may be conjugated to various ligands to facilitate targeting to sebaceous glands or components thereof. These ligands include receptor-specific peptides and/or ligands as well as immunoglobulins and fragments thereof. Non-limiting ligands include antibodies in general and monoclonal antibodies, as well as immunologically reactive fragments of both.

Additional examples of green porphyrin photosensitizers include, but are not limited to, the green porphyrins disclosed in U.S. Pat. Nos. 5,283,255, 4,920,143, 4,883,790, 5,095,030 and 5,171,749, and green porphyrin derivatives discussed in U.S. Pat. Nos. 5,880,145 and 5,990,149. Several structures of typical green porphyrins are shown in the above cited patents, which also provide details for the production of the compounds.

There are a variety of other synthetic and naturally occurring photosensitizers that may be used, including, but not limited to, pro-drugs such as the pro-porphyrin δ-aminolevulinic acid (5-ALA) and derivatives thereof, porphyrins and porphyrin derivatives, e.g., chlorines, bacteriochlorins, isobacyteriochlorins, phthalocyanine and napththalocyanines and other tetra- and poly-macrocyclic compounds, and related compounds (e.g., pyropheophorbides, sapphyrins, and texaphrins) and metal complexes (such as, but not limited to, tin, aluminum, zinc, lutetium). Use of tetrahydrochlorines, purpurins, porphycenes and phenothiaziniums is also contemplated. Other suitable photosensitizers include bacteriochlorophyl derivatives such as those described in WO 97/1981, WO 99/45382 and WO 01/40232. One bacteriochlorophyll is palladium-bacteriopheophorbide WST09 (Tookad™).

A photosensitizer may be a proporphyrin or a porphryin, or a mixture thereof. Some examples of pre-drugs include aminolevulinic acid such a Levulan™ and aminolevulinic acid esters such as described in WO 02/10120 and available as Metvix™, Hexvix™ and Benzvis™. Some examples of di-hydro or tetra-hydro porphyrins are described in EP 0337,601 or WO 01/6650 and available as Foscan™ (temoporfin). Combinations of two or more photosensitizers may be used in the disclosed compositions and methods. A nonexhaustive list of photosensitive chemicals may be found in Kreimer-Birnbaum, Sem. Hematol., 26:157-173 (1989), and in Redmond et al., Photoderm. Photobiol., 70(4):391-475 (1999), both of which are incorporated herein by reference in their entireties.

### Light Energy Administration

Light of a suitable wavelength is applied to the skin to activate the applied photosensitizer. Preferably the light comprises a wavelength close to at least one of the absorption peaks of the photosensitizer. The absorption peaks differ for different photosensitizers. For example, lemuteporfin has an absorption peak at about 688+/−1 nm, and thus, when lemuteporfin is the photosensitizer, the wavelength of light is preferably at or close to about 688+/−1 nm. When the photosensitizer is ALA-methyl ester (Metvix™), which has an absorption peak at 635 nm, the activation energy used is preferably at or close to 635 nm. When the photosensitizer is ALA (available under the trade name Levulan™), which has absorption peaks at 417 nm and 630 nm, the activation energy used is preferably at or close to 417 and/or 630 nm.

The activation or light energy may be provided by any suitable means. Generally, the activation energy is provided by a visible light source. Light energy sources may include, but are not limited to, lasers, light emitting diodes (LED), incandescent lamps, standard fluorescent lamps, U.V. lamps or combinations thereof. Exemplary light sources are light emitting diodes.

Commercially available light sources include CureLight™ (available from Photocure ASA, Oslo, Norway), BLU-U™ (available from DUSA Pharmaceuticals, Wilmington Mass., USA), PDT Laser (available from Diomed, Andover, Mass., USA), Ceralas™ (available from Biolitec AG, Jena, Germany), Omnilux PDT™ (available from PhotoTherapeutics Ltd., Birmingham, UK), and Q-Beam™, SpectraLife™, and Quantamed™ (Quantum Devices Inc., Barneveld, Wis., USA.)

In some embodiments, light is at least in part supplied by light emitting diodes (LEDs). For irradiating a contoured surface such as the face, it may be convenient to use a light source that is configured to follow the contour such as that described in U.S. Pat. No. 7,723,910. PDT for the treatment of acne can be combined with Blu-light Phototherapy in some embodiments of the present disclosure. Therefore some embodiments include light being delivered by an LED device that supplies both red (e.g., 600-750 nm) and blue light (e.g., 390-450 nm). In some cases, a device supplies light at about 420 nm and at about 690 nm.

The dose of light or activation energy administered during a PDT treatment can vary according to the potency of the photosensitizer chosen. For photosensitizers of high potency, such as green porphyrins, the dosage of light is in the range of about 5 to about 400 J/cm², or more preferably in the range of about 25 to about 300 J/cm², as non-limiting examples. In some embodiments, the light dose used in PDT treatment is in the range of about 25 to about 50 J/cm², about 50 to about 100 J/cm², about 100 to about 150 J/cm², about 150 to about 200 J/cm², about 200 to about 250 J/cm², about 250 to about 300 J/cm², about 300 to about 350 J/cm², about 350 to about 400 J/cm², about 400 to 450 J/cm², about 450 to about 500 J/cm², about 500 to about 550 J/cm², or about 550 to 600 J/cm². Other non-limiting examples of light doses include doses of about 25, about 50, about 75, about 100, about 125, about 150, about 175, about 200, about 250 or about 300 J/cm².

The total light dose depends upon the intensity of the radiation source (also known as the fluence rate or irradiance) and the time of irradiation. Once the total dose of radiation is chosen, the fluence rate can be adjusted so that the treatment can be completed in a reasonable period of time. The period of irradiation or light exposure typically lasts from about 10 seconds to about 4 hours. For green porphyrins such as lemuteporfin, the light exposure typically lasts between 1 minute and 2 hours, more preferably between about 5 minutes and about 60 minutes. Some exemplary irradiation times are about 1, about 5, about 10, about 15, about 25, about 30, about 35, about 40, about 45, about 50, about 55 or about 60 minutes.

The intensity of the energy or light source is generally below 600 mW/cm². In certain aspects, irradiances may be between about 10 and 500 mW/cm². In other embodiments according to the present disclosure, the irradiance may be between about 25 and about 100 mW/cm². In some embodiments, the irradiance is 50 mW/cm². In other embodiments, the irradiance is 80 mW/cm². In other embodiments, the light dose is varied between 37.5 J/cm² and 150 J/cm² by varying the time of irradiation at a fixed fluence rate of 80 mW/cm² between 7 minutes, 49 seconds to 31 minutes, 15 seconds.

### PDT Treatment of Acne and Other Hyperactive Sebaceous Gland Conditions

The present disclosure also includes and provides methods for treating a hyperactive sebaceous gland disorder in an affected area of the skin of a subject in need thereof, comprising topically applying a therapeutically effective amount of a photosensitizer composition of the present disclosure to the affected area of the skin of the subject, allowing sufficient time for at least some of the photosensitizer to localize in the sebaceous glands, and exposing the skin of the subject to light energy at a wavelength capable of activating the photosensitizer. In some embodiments, the hyperactive sebaceous gland disorder is acne (including acne vulgaris), seborrhea (or oily skin), seborrheic dermatitis, hidradenitis suppurativa (acne inverse), and sebaceous gland hyperplasia. In some embodiments, the subjects have both acne and oily skin.

The present disclosure also includes and provides methods for reducing sebum production by sebaceous glands of a subject in need thereof, comprising topically applying a therapeutically effective amount of a photosensitizer composition of the present disclosure to the affected skin of a subject in need of treatment, allowing sufficient time for at least some of the photosensitizer to localize in the sebaceous glands, and exposing the skin of the subject to light energy at a wavelength capable of activating the photosensitizer, whereby the sebum excretion rate of the subject is reduced.

The present disclosure also includes and provides methods of treating acne in a subject in need thereof, comprising topically applying a therapeutically effective amount of a photosensitizer composition of the present disclosure, allowing sufficient time for at least some of the photosensitizer to localize in the sebaceous glands of the subject, and exposing the skin of the subject to light energy at a wavelength capable of activating the photosensitizer.

The present disclosure also includes and provides methods for ablating sebocytes in a subject afflicted with a hyperactive sebaceous gland disorder such as acne, comprising the steps of delivering a therapeutically effective amount of a photosensitizer to the sebocytes of the subject, allowing sufficient time for the photosensitizer to localize in the sebocytes, and exposing the sebocytes to light energy at a wavelength capable of activating the photosensitizer.

Conditions that may be treated include any condition for which a topical formulation of a photosensitizer is suitable. Non-limiting examples include skin conditions such as dermatitis, psoriasis, malignant and pre-malignant skin lesions, actinic keratosis, and hyperactive sebaceous gland disorders. Hyperactive sebaceous gland disorders include, without limitation, acne (including acne vulgaris), seborrhea (or oily skin), seborrheic dermatitis, hidradenitis, suppurativa, and sebaceous gland hyperplasia. Any part of the body may be treated, but conditions such as acne and oily skin typically affect the face, chest and/or back.

For a PDT treatment, the skin is first preferably washed with an antibacterial cleanser and dried. The skin may be treated with dry heat (IR) until either the skin temp reaches 45° C. or for a fixed time such as 20 min. This may enhance the penetration of photosensitizer into the sebaceous glands. Alternatively, the skin may also be treated with microderm abrasion. The skin may be degreased (e.g. using acetone or isopropyl alcohol) if necessary before application of the photosensitizer.

Once this skin surface has been cleansed and prepared, the chosen formulation of photosensitizer is applied to the affected area of a skin surface after the area has been thoroughly cleansed. The photosensitizer-containing formulation is left in contact with the skin for sufficient time to allow the photosensitizer to localize in the sebaceous glands of the subject. Generally the time of contact could be between about 1 minute and about 24 hours or longer, depending on the type and concentration of the photosensitizer in the formulation. Preferably, the formulation is in contact with the skin for about 1 to about 180 minutes if the photosensitizer is a green porphyrin such as lemuteporfin. Exemplary contact times are about 1, about 5, about 10, about 20, about 30, about 40, about 50, about 60, about 70, about 80, about 90, about 100, about 110, about 120, about 130, about 140, about 150, about 160, about 170 or about 180 minutes. Additional exemplary contact times are about 3.5, about 4, about 4.5, about 5, about 5.5, about 6, about 6.5, about 7, about 7.5 or about 8 hours. Excess formulation is then preferably removed with clean gauze or cloth moistened with lukewarm water. Irradiation is then applied as described above. It may be advisable to use a regimen of increasing light dose until the subject maximum tolerated dose (MTD) is determined. Pain at the site of irradiation or erythema following PDT are signs that the MTD has been exceeded. Thereafter, the person may be treated at or below the MTD.

The treatment may be repeated as many times as necessary to have a therapeutic effect. If repeated, the treatment frequency may vary. For example, the treatments could be daily, about every two days, about twice weekly, about weekly, about every two weeks, about twice monthly, about every four week, about monthly, about every six weeks, about every eight weeks, about every two months, about quarterly, about twice annually, or about annually, or other suitable time interval. In certain aspects, the treatment interval is every two weeks to every six months. Treatment can continue until the desired degree of improvement in the skin condition has occurred. For example, treatments may be repeated until the total number of acne lesions is reduced by about 5%, about 10%, about 15%, about 20%, about 25%, about 30%, about 40%, about 50%, about 60%, about 70%, about 80% or about 90% or more. To take another non-limiting example, treatments may be repeated until the sebum excretion rate has been reduced by about 5%, about 10%, about 15%, about 20%, about 25%, about 30%, about 40%, about 50%, about 60%, about 70%, about 80% or about 90% or more.

### Determining Efficacy of Treatment

The efficacy of the disclosed compositions and methods may be determined by any suitable means. In many cases, a simple decrease, reduction, or improvement in the sebaceous gland disorder or other skin disorder, as recognized by a skilled physician may be used to determine efficacy. Thus an improvement in a hyperactive sebaceous gland disorder, such as an improvement in a subject's acne, seborrhea, seborrheic dermatitis, hidradenitis suppurativa, or sebaceous gland hyperplasia, may be used as an indication of efficacy.

Taking acne as a non-limiting example, efficacy may be determined based upon quantitative and/or qualitative data. The total number of lesions can be assessed by predefining one or more test areas before commencement of treatment. Lesion counts (non-inflammatory, inflammatory and total, or open comedones, closed comedones, papules, pustules and nodules) are performed within the test area before and after treatment. Sizes of the lesions within the test area are also recorded. The test areas are also photographed. A number of test areas may be selected for each subject and the location of the test area may vary depending on the locale of the acne lesions of the subject. The test areas may be assessed within the first week, after one week, after two weeks, or after a month or two of the initial PDT treatment, or at other desired frequencies. A global assessment scale such as the 5-point Investigator Global Assessment (IGA) for acne vulgaris, as recommended by the FDA and shown in Table 2 may be used to measure efficacy.

TABLE 2
Investigator's Global Assessment (IGA) Scale

| Score | Description |
|---|---|
| 0 | No acne: No evidence of acne vulgaris |
| 1 | Minimal: Few non-inflammatory lesions (comedones) are |
| | present; a few inflammatory lesions (papules/pustules) may |
| | be present) |
| 2 | Mild: Several to many non-inflammatory lesions (comedones) |
| | are present; a few inflammatory lesions (comedones) are |
| | present |
| 3 | Moderate: Many non-inflammatory (comedones) and |
| | inflammatory (papules/pustules) are present; no nodulocystic |
| | lesions are present |
| 4 | Severe: Significant degree of inflammatory disease; |
| | papules/pustules are a predominant feature; a few |
| | nodulocystic lesions may be present; comedones may be |
| | present |

The efficacy of PDT for reducing sebum production may be measured by using SebuTape™, a product designed specifically for that purpose and available from CuDerm Corporation, Dallas, Tex., USA. Example 9 herein demonstrates how to use SebuTape™ to obtain an accurate measurement of sebum exudation. SebuTape™ measurements may be done within the first week, after one week, after two weeks, or after a month or two of the initial PDT treatment, or at other desired frequencies. The efficacy of PDT for reducing the number of sebaceous glands may be measured by taking biopsies following PDT treatment, and using histology with Oil Red O staining to determine the total number of PSU (hair follicles structures with or without sebaceous glands) in an image followed by a count of the number of lipid-staining (sebaceous gland containing) staining PSU. This procedure is described in Example 3 herein.

### EXAMPLES

### Example 1

### Solubility of Lemuteporfin in Various Solvents

The solubility of lemuteporfin in various solvent compositions is shown in the last column of Table 3. All values were obtained analytically by HPLC analysis.

Solubility results for lemuteporfin indicated maximum solubility in solvent-based formulations containing primarily benzyl alcohol. The amount of lemuteporfin that can be solubilized in benzyl alcohol following heating is approximately 2.5% w/w. The addition of other solvents reduces solubility by approximately the amount of the new solvent introduced. Diethylene glycol monoethyl ether (DGME) is about 20% as efficient at dissolving lemuteporfin as benzyl alcohol.

TABLE 3
Apparent Solubility of Lemuteporfin in Various Solvents

| | Benzyl | Oleyl | Oleic | Propylene | Ethanol | Isopropyl | % w/w |
|---|---|---|---|---|---|---|---|
| DGME* | Alcohol | Alcohol | Acid | Glycol | (190) | Myristate | Lemuteporfin |
| 65 | — | 35 | — | — | — | — | 0.430 |
| 65 | — | 17.5 | 17.5 | — | — | — | 0.271 |
| 50 | 15 | — | 35 | — | — | — | 0.230 |
| 50 | 15 | 35 | — | — | — | — | 0.200 |
| 52 | 16 | 16 | 16 | — | — | — | 0.210 |
| — | 30 | — | — | — | 70 | — | 0.455 |
| — | 100 | — | — | — | — | — | 2.55 |
| — | 30 | 70 | — | — | — | — | 0.166 |
| 50 | 50 | — | — | — | — | — | 1.57 |
| — | 50 | — | — | 50 | — | — | 0.494 |
| — | 50 | — | — | — | 50 | — | 0.453 |
| — | 50 | 50 | — | — | — | — | 0.228 |
| — | 50 | — | 50 | — | — | — | 0.409 |
| — | 40 | — | — | — | — | 60 | 0.090 |
| 90 | — | 10 | — | — | — | — | 0.420 |
| 90 | — | 5 | 5 | — | — | — | 0.420 |
| 90 | — | — | — | — | — | 10 | 0.430 |
| — | 90 | 10 | — | — | — | — | 2.24 |
| — | 90 | — | 10 | — | — | — | 2.33 |
| — | 90 | 5 | 5 | — | — | — | 2.22 |
| — | 90 | — | — | — | — | 10 | 2.30 |
| 45 | 45 | 5 | 5 | — | — | — | 1.373 |

*values under solvent name are % w/w of solvent in solution composition

### Example 2

### The Effect of Viscosity-Enhancing Agents on Photosensitizer Formulations

To assess the impact of increasing the viscosity of lemuteporfin formulations on the efficacy of PDT to ablate mouse sebaceous glands, photosensitizer compositions were prepared with the components shown in Table 4 and applied onto shaved mouse flank skin for 30 minutes prior to exposure with 688 nm red light (50 J/cm² or 100 J/cm² delivered at a rate of 50 mW/cm²). Each treatment group consisted of 5 animals.

TABLE 4

| Component (w/w %) | LT-G-001 | LT-G-002 | LT-G-003 | LT-G-004 | LT-G-005 | LTO-TG1 |
|---|---|---|---|---|---|---|
| lemuteporfin | 0.1 | 0.1 | 0.1 | 0.1 | 0.02 | 0.2 |
| benzyl alcohol | 5 | 5 | 5 | 5 | 5 | — |
| isopropyl alcohol | 48.9 | 48.9 | 48.9 | 48.9 | 48.9 | — |
| diethylene glycol | 32 | 32 | 32 | 32 | 32 | 20.0 |
| monomethyl ether | | | | | | |
| oleyl alcohol | 5 | 5 | 5 | 5 | 5 | 10.0 |
| methyl salicylate | 1 | 1 | 1 | 1 | 1 | — |
| polysorbate 80 | 0.5 | 0.5 | 0.5 | 0.5 | 0.5 | — |
| menthol | 2.5 | 2.5 | 2.5 | 2.5 | 2.5 | — |
| hydroxy-propyl | 4 | — | 2 | 4 | 4 | — |
| cellulose | | | | | | |
| ethyl cellulose | 1 | — | | | 1 | — |
| PEG400 | — | — | — | — | — | 53.8 |
| PEG3350 | — | — | — | — | — | 16.0 |

To assess sebaceous gland changes, mice were sacrificed 72 hours after PDT. Full-thickness skin from within the tattoo points on the PDT-treated right flank was carefully excised. The upper half of these tissue squares was placed in a plastic mold filled with Neg-50™ cryo embedding medium and frozen on liquid nitrogen. The lower half was preserved in formol acetic alcohol for 18 hours. The tissue was transferred to 70% alcohol until processed to wax by a standard in-house protocol. Formalin-fixed samples were subsequently stained with standard reagents (e.g., hematoxylin and eosin) to assess general histological changes within the tissue if required.

For sebaceous gland evaluations, frozen tissue samples were cut in 8 μm sections with a cryostat onto glass slides and immediately fixed in 10% buffered formalin. Three sets of 2 slides were cut from each block with the distance between sets of approximately 200 μm. One slide from each set was stained with Oil Red O and then cover-slipped with acrylic mounting medium and allowed to set. The second slide from each set was used as a “back-up” in the event that the first slide is damaged.

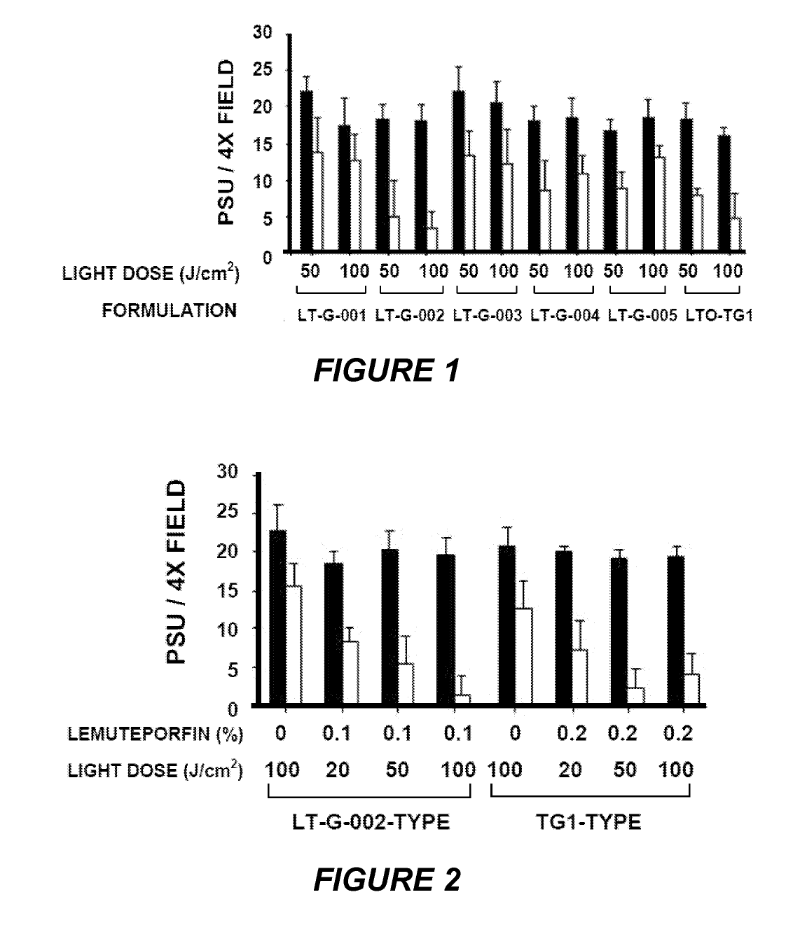

Images were taken of representative sections from each cross-section using a 4× objective mounted on an Olympus BX61 microscope fitted with a digital camera. Slides were assessed by counting the total number of PSU (hair follicles structures with or without sebaceous glands) in an image followed by a count of the number of lipid-staining (sebaceous gland containing) staining PSU. Slides were evaluated by two independent readers. The results are shown in FIG. 1.

Due to the large number of mice required to test a matched-vehicle for each formulation, no control groups are included in this experiment. However, typically, for naïve mouse flank skin 70-80% of PSU contained detectable Oil Red O-positive sebaceous glands. The most effective composition in producing the lowest number of PSU with Oil Red O-positive sebaceous glands is formulation LT-G-002 (FIG. 1). This formulation did not contain a viscosity modifying agent. On average, approximately 30% of PSU in flank skin treated with LT-G-002 and either light dose contains Oil Red O-positive glands. PDT with LTO-TG1 has a similar, but somewhat lower, reductive effect on sebaceous gland counts. In contrast, sebaceous gland counts for mice treated with PDT using Formulations LT-G-001, LT-G-003, LT-G-004 or LT-G-005 (all containing a viscosity enhancing agent that was either hydroxy-propyl cellulose, ethyl cellulose, or both) are not nearly as effective. Thus, such viscosity enhancing agents may prevent the localization of the lemuteporfin in sebaceous glands.

### Example 3

### Effect of Different PDT Light Doses on Mouse Sebaceous Glands with a Lemuteporfin Composition Lacking a Viscosity Enhancing Agent

This experiment compares the effect of PDT with LT-G-002 to that of LTO-TG1 (which contains twice the amount of lemuteporfin) at three different red light doses. PDT with either lemuteporfin topical formulation affected sebaceous counts with reductive effects at red light doses of 20, 50 or 100 J/cm² as compared to the result obtained for mice treated with control vehicle and a red light dose of 100 J/cm² (FIG. 2). Greater effects on gland counts, with either lemuteporfin formulation, were produced with red light doses of 50 and 100 J/cm² than at 20 J/cm².

### Example 4

### Lemuteporfin Localization in Human Hair Follicles and Sebaceous Glands

The model for localization of lemuteporfin in human skin utilized dermatomed human cadaver skin procured from Ohio Valley Tissue Bank, fresh (≦24 hours post-mortem) and human skin procured from NDRI (National Disease Research Interchange). This experiment compared a lemuteporfin topical solution (LTS) without a viscosity enhancing agent to a lemuteporfin topical ointment (LTO) (LTO-TG1 from Example 2, Table 4). The LTS formulation included lemuteporfin, 0.1%, oleyl alcohol, 5%, benzyl alcohol 5%, DGME 32%, Vitamin E TPGS, 0.5%, menthol, 5%, and ethanol, 52% all w/w. The formulations were applied to the skin in a measured amount and left open to the air. The skin was maintained in contact with the formulations for the designated period of time (1 or 8 hours), biopsied, set in Neg-50™ frozen tissue medium and then prepared for sectioning and fluorescence microscopy evaluation.

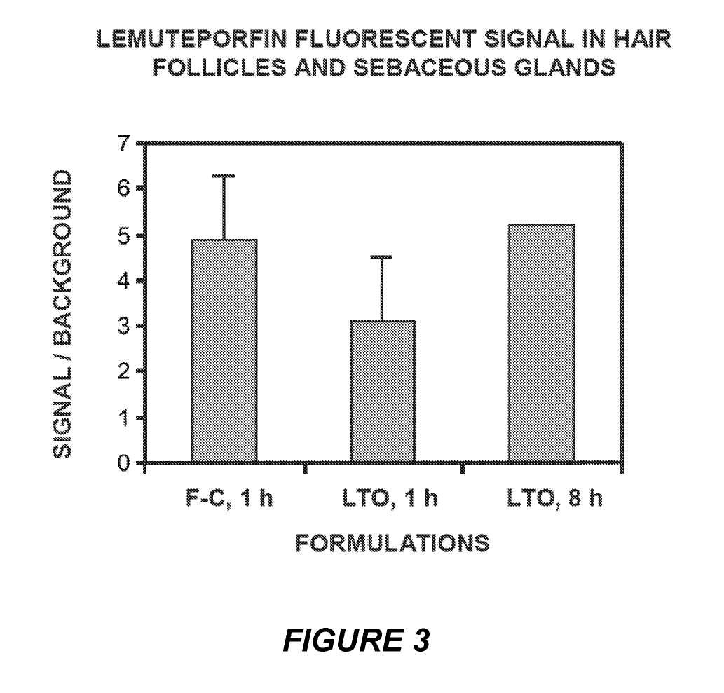

The tissue fluorescence results showed that the LTS formulation localized in human cadaver skin sebaceous glands within one hour to an extent that it requires 8 hours for an LTO formulation containing twice the amount of lemuteporfin to achieve (FIG. 3). Thus a solution type of formulation provided more rapid delivery of lemuteporfin to human sebaceous glands than an ointment form. This is important in a clinical setting in which a subject must wait for a specified period of time between application of a photosensitizer-containing formulation and activation of the photosensitizer with light: a shorter period of time is better.

### Example 5

### Stability of LTS Photosensitizer Composition

A batch of lemuteporfin topical solution was prepared according to the formula in Table 5, dispensed into 5 ml vials, and maintained for stability testing. After three months, precipitation was observed in some vials. The precipitate was identified as lemuteporfin by standard analytical techniques. An optimal delivery system for lemuteporfin would contain a relatively high concentration of lemuteporfin, but also must contain components in which lemuteporfin is not readily soluble such as DGME (see Example 1). Thus it was necessary to implement a different approach to formulating lemuteporfin if long term storage is desired.

TABLE 5
A Lemuteporfin Topical Solution (LTS)

| Formulation | Percentage |
|---|---|
| Component | w/w |
| Lemuteporfin | 0.1% |
| Oleyl Alcohol | 5% |
| Benzyl Alcohol | 10% |
| DGME | 32% |
| Polysorbate 80 | 0.5% |
| Menthol | 2.5% |
| Isopropanol | 48.9% |
| Methyl Salicylate | 1.0 |

### Example 6

### Solubility Studies of Formulated Lemuteporfin

Lemuteporfin topical solution (LTS) was prepared by adding lemuteporfin to the other components in Table 5 (already pre-mixed) at room temperature. The solution was stirred and samples were removed at various time points, and then filtered to determine the amount of undissolved lemuteporfin. The results are presented in Table 6. The amount of lemuteporfin that dissolved was approximately 0.048%.

It was possible to manufacture a lemuteporfin topical solution 0.1% by dissolving the drug in DGME and benzyl alcohol at high temperature, approximately 75° C. The solution was then cooled to room temperature and the remaining LTS components were added and mixed to form a homogenous solution. Based on the solubility data, this manufacturing process resulted in a supersaturated solution.

TABLE 6
Lemuteporfin Solubility in the Formulation
Shown in Table 5

| | Lemuteporfin content |
|---|---|
| Time point (hours) | (% w/w) |
| 0.17 | 0.0241 |
| 2.47 | 0.0482 |
| 4.37 | 0.0453 |
| 6.37 | 0.0463 |
| 23.5 | 0.0370 |

Studies were conducted to determine the effect of certain of the LTS excipients on the solubility of lemuteporfin. Removing isopropyl alcohol from the solution system increased the solubility of lemuteporfin from approximately 0.03% to 0.07% w/w (data not shown). Polysorbate 80 increased the solubility from 0.027% to 0.037% w/w (data not shown).

### Example 7

### Two-Component Formulation System

To solve the problems of the solubility and long-term stability of lemuteporfin in an effective topical delivery formulation, a two-component formulation system was developed. The first component is the photosensitizer component comprising lemuteporfin dissolved in a solvent in which it is most soluble. The second component is the diluent component comprising the remainder of the LTS excipients. Some examples of LTS two-component formulations are shown in Tables 7 to 24.

The compositions described in Tables 7 to 24 were made as follows. The photosensitizer component (lemuteporfin-containing) and diluent component were manufactured in separate compounding vessels. A jacketed beaker connected to a water bath was set at 75° C. and placed on a stir plate. The photosensitizer component was mixed while being heated for approximately 1 hour. After one hour of heating, the active solution was cooled to room temperature while mixing was continued. The diluent excipients were weighed and transferred to a separate glass vessel. The diluent excipients were mixed at room temperature for approximately 30-60 minutes.

Filling was performed using a Flexicon® vial filler. Fill checks were performed and the average fill weight was within the 2% of the target fill weight. The diluent component was filled first, followed by the photosensitizer component for each batch manufacture. After filling, all vials were labeled and then placed in a USP controlled room temperature or a desired temperature (e.g., at 2-8° C.).

TABLE 7
Batch A (0.1% w/w)

| | | | Batch |
|---|---|---|---|
| | | | Formula |
| Vial | Component | % w/w | Weight (g) |
| 1 | Lemuteporfin | 0.1 | 0.5001 |
| | Diethylene glycol | 32 | 160.1939 |
| | monoethyl ether | | |
| | (DGME) | | |
| | Benzyl alcohol | 5 | 25.0829 |
| 2 | Isopropanol (IPA) | 53.9 | 269.5 |
| | Oleyl alcohol | 5 | 25.0595 |
| | Polysorbate 80 | 0.5 | 2.5141 |
| | Methyl salicylate | 1 | 5.0144 |
| | Menthol | 2.5 | 12.5040 |
| | Total weight | 100 | 500.3689 |

TABLE 8
Batch B (0.075% w/w)

| | | | Batch |
|---|---|---|---|
| | | | Formula |
| Vial | Component | % w/w | Weight (g) |
| 1 | Lemuteporfin | 0.075 | 0.3751 |
| | Diethylene glycol | 32 | 160.0135 |
| | monoethyl ether | | |
| | (DGME) | | |
| | Benzyl alcohol | 5 | 25.0653 |
| 2 | Isopropanol (IPA) | 53.925 | 269.6259 |
| | Oleyl alcohol | 5 | 25.0114 |
| | Polysorbate 80 | 0.5 | 2.5017 |
| | Methyl salicylate | 1 | 5.0016 |
| | Menthol | 2.5 | 12.5091 |
| | Total weight | 100 | 500.1036 |

TABLE 9
Batch C (0.1% w/w) - Formulation Lemuteporfin TK1

| | Target | | | | |
|---|---|---|---|---|---|
| | Fill | | | | Batch Formula |
| Vial | Weight | | Component | % w/w | Weight (g) |
| 1 | 0.9 | g | Lemuteporfin | 0.1 | 1.0005 |
| | | | Benzyl alcohol | 10 | 100.1729 |
| 2 | 8.01 | g | Diethylene glycol | 32 | 320.1 |
| | | | monoethyl ether | | |
| | | | (DGME) | | |
| | | | Isopropanol (IPA) | 48.9 | 490.0 |
| | | | Oleyl alcohol | 5 | 50 |
| | | | Polysorbate 80 | 0.5 | 5 |
| | | | Methyl salicylate | 1 | 10.1 |
| | | | Menthol | 2.5 | 25.0 |
| | | | Total weight | 100 | 1001.3734 |

TABLE 10
Batch D (0.075% w/w) - Formulation Lemuteporfin TK2

| | Target | | | | |
|---|---|---|---|---|---|
| | Fill | | | | Batch Formula |
| Vial | Weight | | Component | % w/w | Weight (g) |
| 1 | 0.9 | g | Lemuteporfin | 0.075 | 0.7507 |
| | | | Benzyl alcohol | 10 | 100.02 |
| 2 | 8.03 | g | Diethylene glycol | 32 | 320 |
| | | | monoethyl ether | | |
| | | | (DGME) | | |
| | | | Isopropanol (IPA) | 48.925 | 489.9 |
| | | | Oleyl alcohol | 5 | 50.1 |
| | | | Polysorbate 80 | 0.5 | 5.1 |
| | | | Methyl salicylate | 1 | 10.5 |
| | | | Menthol | 2.5 | 25.1 |
| | | | Total weight | 100 | 1001.4707 |

TABLE 11
Batch H (0.05% w/w) - Formulation Lemuteporfin TK3

| | Target | | | Batch |
|---|---|---|---|---|
| | Fill | | | Formula |
| Vial | Weight | Component | % w/w | Weight (g) |
| 1 | 0.9 g | Lemuteporfin | 0.05 | 0.5001 |
| | | Benzyl alcohol | 10 | 100.3 |
| 2 | 8.0 g | Diethylene glycol | 32 | 320.1 |
| | | monoethyl ether | | |
| | | (DGME) | | |
| | | Isopropanol (IPA) | 48.95 | 489.9 |
| | | Oleyl alcohol | 5 | 50.0 |
| | | Polysorbate 80 | 0.5 | 5.1 |
| | | Methyl salicylate | 1 | 10.1 |
| | | Menthol | 2.5 | 25.1 |
| | | Total weight | 100 | 1001.1 |

TABLE 12
Batch 1 (0.12% w/w) - Formulation Lemuteporfin P2

| | Target | | | | |
|---|---|---|---|---|---|
| | Fill | | | | Amount per |
| Vial | Weight | | Component | % w/w | vial (g) |
| 1 | 0.9 | g | Lemuteporfin | 0.12 | 0.009 |
| | | | Benzyl alcohol | 11.9 | 0.891 |
| 2 | 6.62 | g | Diethylene glycol | | |

| | | |
|---|---|---|
| monoethyl ether | 22.5 | 1.692 |
| (DGME) | | |
| Isopropanol (IPA) | 58.0 | 4.360 |
| Oleyl alcohol | 5.9 | 0.450 |
| Polysorbate 80 | 0.6 | 0.040 |
| Methyl salicylate | 0.9 | 0.072 |
| Menthol | 0.06 | 0.004 |
| Total weight | 100 | 7.52 |

TABLE 13
Batch J (0.12% w/w) - Formulation Lemuteporfin P3

| | Target | | | | |
|---|---|---|---|---|---|
| | Fill | | | | Amount per |
| Vial | Weight | | Component | % w/w | vial (g) |
| 1 | 0.9 | g | Lemuteporfin | 0.12 | 0.009 |
| | | | Benzyl alcohol | 12.0 | 0.891 |
| 2 | 6.54 | g | Diethylene glycol | 22.8 | 1.692 |

| | | |
|---|---|---|
| monoethyl ether | | |
| (DGME) | | |
| Isopropanol (IPA) | 58.6 | 4.360 |
| Oleyl alcohol | 6.0 | 0.450 |
| Polysorbate 80 | 0.6 | 0.040 |
| Methyl salicylate | 0 | 0 |
| Menthol | 0 | 0 |
| Total weight | 100 | 7.44 |

TABLE 14
Batch K (0.13% w/w) - Formulation Lemuteporfin P5

| | Target | | | | |
|---|---|---|---|---|---|
| | Fill | | | | Amount per |
| Vial | Weight | | Component | % w/w | vial (g) |
| 1 | 0.9 | g | Lemuteporfin | 0.13 | 0.009 |
| | | | Benzyl alcohol | 12.8 | 0.891 |
| 2 | 6.05 | g | Diethylene glycol | 24.4 | 1.692 |

| | | |
|---|---|---|
| monoethyl ether | | |
| (DGME) | | |
| Isopropanol (IPA) | 62.7 | 4.360 |
| Oleyl alcohol | 0 | 0 |
| Polysorbate 80 | 0 | 0 |
| Methyl salicylate | 0 | 0 |
| Menthol | 0 | 0 |
| Total weight | 100 | 6.95 |

TABLE 15
Batch L (0.34% w/w) - Formulation Lemuteporfin PX

| | Target | | | |
|---|---|---|---|---|
| | Fill | | | Amount per |
| Vial | Weight | Component | % w/w | vial (g) |
| 1 | 1.29 g | Lemuteporfin | 0.34 | 0.009 |
| | | Benzyl alcohol | 48.76 | 1.281 |
| 2 | 1.34 g | Diethylene glycol | 16.94 | 0.445 |
| | | monoethyl ether | | |
| | | (DGME) | | |
| | | Isopropanol (IPA) | 33.96 | 0.892 |
| | | Oleyl alcohol | 0 | 0 |
| | | Polysorbate 80 | 0 | 0 |
| | | Methyl salicylate | 0 | 0 |
| | | Menthol | 0 | 0 |
| | | Total weight | 100 | 2.63 |

TABLE 16
Batch M (0.40% w/w) Formulation Lemuteporfin P12

| | Target | | | | |
|---|---|---|---|---|---|
| | Fill | | | | Amount per |
| Vial | Weight | | Component | % w/w | vial (g) |
| 1 | 0.9 | g | Lemuteporfin | 0.40 | 0.009 |
| | | | Benzyl alcohol | 39.8 | 0.891 |
| 2 | 1.34 | g | Diethylene glycol | 20 | 0.445 |

| | | |
|---|---|---|
| monoethyl ether | | |
| (DGME) | | |
| Isopropanol (IPA) | 39.8 | 0.892 |
| Oleyl alcohol | 0 | 0 |
| Polysorbate 80 | 0 | 0 |
| Methyl salicylate | 0 | 0 |
| Menthol | 0 | 0 |
| Total weight | 100 | 2.24 |

TABLE 17
Batch N (0.50% w/w) Formulation Lemuteporfin P14

| | Target | | | | |
|---|---|---|---|---|---|
| | Fill | | | | Amount per |
| Vial | Weight | | Component | % w/w | vial (g) |
| 1 | 0.9 | g | Lemuteporfin | 0.50 | 0.009 |
| | | | Benzyl alcohol | 49.8 | 0.891 |
| 2 | 0.89 | g | Diethylene glycol | 24.9 | 0.445 |

| | | |
|---|---|---|
| monoethyl ether | | |
| (DGME) | | |
| Isopropanol (IPA) | 24.9 | 0.446 |
| Oleyl alcohol | 0 | 0 |
| Polysorbate 80 | 0 | 0 |
| Methyl salicylate | 0 | 0 |
| Menthol | 0 | 0 |
| Total weight | 100 | 1.80 |

TABLE 18
Batch O (0.40% w/w) Formulation Lemuteporfin P15

| | Target | | | | |
|---|---|---|---|---|---|
| | Fill | | | | Amount per |
| Vial | Weight | | Component | % w/w | vial (g) |
| 1 | 0.9 | g | Lemuteporfin | 0.40 | 0.009 |
| | | | Benzyl alcohol | 39.1 | 0.891 |
| 2 | 1.38 | g | Diethylene glycol | 19.5 | 0.445 |

| | | |
|---|---|---|
| monoethyl ether | | |
| (DGME) | | |
| Isopropanol (IPA) | 31.3 | 0.713 |
| Acetone | 9.8 | 0.223 |
| Oleyl alcohol | 0 | 0 |
| Polysorbate 80 | 0 | 0 |
| Methyl salicylate | 0 | 0 |
| Menthol | 0 | 0 |
| Total weight | 100 | 2.28 |

TABLE 19
Batch P (0.355% w/w) Formulation Lemuteporfin P16

| | Target | | | |
|---|---|---|---|---|
| | Fill | | | Amount per |
| Vial | Weight | Component | % w/w | vial (g) |
| 1 | 1.20 g | Lemuteporfin | 0.355 | 0.009 |
| | | Benzyl alcohol | 46.95 | 1.191 |
| 2 | 1.34 g | Diethylene glycol | 17.54 | 0.445 |
| | | monoethyl ether | | |
| | | (DGME) | | |
| | | Isopropanol (IPA) | 35.16 | 0.892 |
| | | Oleyl alcohol | 0 | 0 |
| | | Polysorbate 80 | 0 | 0 |
| | | Methyl salicylate | 0 | 0 |
| | | Menthol | 0 | 0 |
| | | Total weight | 100 | 2.54 |

TABLE 20
Batch Q (0.375% w/w) Formulation Lemuteporfin P17

| | Target | | | |
|---|---|---|---|---|
| | Fill | | | Amount per |
| Vial | Weight | Component | % w/w | vial (g) |
| 1 | 1.20 g | Lemuteporfin | 0.375 | 0.009 |
| | | Benzyl alcohol | 49.63 | 1.191 |
| 2 | 1.20 g | Diethylene glycol | 16.67 | 0.400 |
| | | monoethyl ether | | |
| | | (DGME) | | |
| | | Isopropanol (IPA) | 33.33 | 0.800 |
| | | Oleyl alcohol | 0 | 0 |
| | | Polysorbate 80 | 0 | 0 |
| | | Methyl salicylate | 0 | 0 |
| | | Menthol | 0 | 0 |
| | | Total weight | 100 | 2.40 |

TABLE 21
Batch R (0.354% w/w) Formulation Lemuteporfin P18

| | Target | | | |
|---|---|---|---|---|
| | Fill | | | Amount per |
| Vial | Weight | Component | % w/w | vial (g) |
| 1 | 1.20 g | Lemuteporfin | 0.354 | 0.009 |
| | | Benzyl alcohol | 46.888 | 1.191 |
| 2 | 1.20 g | Diethylene glycol | 17.717 | 0.450 |
| | | monoethyl ether | | |
| | | (DGME) | | |
| | | Isopropanol (IPA) | 35.040 | 0.890 |
| | | Oleyl alcohol | 0 | 0 |
| | | Polysorbate 80 | 0 | 0 |
| | | Methyl salicylate | 0 | 0 |
| | | Menthol | 0 | 0 |
| | | Total weight | 100 | 2.54 |

Table 22
Batch S (0.354% w/w) Formulation Lemuteporfin F20

| | Target | | | % w/w | Amount | |
|---|---|---|---|---|---|---|
| | Fill | | | (Final | per vial^{a} | % w/w |
| Vial | Weight^{a} | | Component | mixed) | (g) | per vial |
| 1 | 0.9 | g^{a} | Lemuteporfin | 0.354% | 0.009 | 1.0% |
| | | | Benzyl alcohol | 46.9% | 0.891 | 99.0% |
| 2 | 1.64 | g^{a} | Benzyl alcohol | | 0.300 | 18.3% |

| | | | |
|---|---|---|---|
| Diethylene glycol | 17.7% | 0.450 | 27.4% |
| monoethyl ether | | | |
| (DGME) | | | |
| Isopropanol (IPA) | 35.0% | 0.890 | 54.3% |
| Oleyl alcohol | 0 | 0 | |
| Polysorbate 80 | 0 | 0 | |
| Methyl salicylate | 0 | 0 | |
| Menthol | 0 | 0 | |
| TOTAL | 100.0% | 2.54^{a} | |

^{a}Weight as shown or multiples of thereof keeping Vial 1 and Vial 2 contents in the same ratios for a total weights of 6.0, 9.0, 12.0, 15.0 g, etc.

TABLE 23
Batch T (0.3% w/w) Formulation Lemuteporfin F21

| | Target | | | % w/w | Amount | |
|---|---|---|---|---|---|---|
| | Fill | | | (Final | per vial^{a} | % w/w |
| Vial | Weight^{a} | | Component | mixed) | (g) | per vial |
| 1 | 0.9 | g^{a} | Lemuteporfin | 0.3% | 0.009 | 0.6 |
| | | | Benzyl alcohol | 49.7% | 1.491 | 99.4 |
| 2 | 2.10 | g^{a} | Benzyl alcohol | 0% | 0 | 0 |

| | | | |
|---|---|---|---|
| Diethylene glycol | 17% | 0.510 | 34 |
| monoethyl ether | | | |
| (DGME) | | | |
| Isopropanol (IPA) | 33% | 0.990 | 66 |
| Oleyl alcohol | 0 | 0 | |
| Polysorbate 80 | 0 | 0 | |
| Methyl salicylate | 0 | 0 | |
| Menthol | 0 | 0 | |
| TOTAL | 100.0% | 3.000^{a} | |

^{a}Weight as shown or multiples of thereof keeping Vial 1 and Vial 2 contents in the same ratios for a total weights of 6.0, 9.0, 12.0, 15.0 g, etc.

TABLE 24
Batch U (0.3% w/w) Formulation Lemuteporfin F21

| | Target | | | % w/w | Amount | |
|---|---|---|---|---|---|---|
| | Fill | | | (Final | per vial ^{a} | % w/w |
| Vial | Weight ^{a} | | Component | mixed) | (g) | per vial |
| 1 | 0.9 | g ^{a} | Lemuteporfin | 0.3% | 0.009 | 1 |
| | | | Benzyl alcohol | 49.7% | 0.891 | 99 |
| 2 | 2.10 | g ^{a} | Benzyl alcohol | | 0.600 | 28.55 |

| | | | |
|---|---|---|---|
| Diethylene glycol | 17% | 0.510 | 24.3 |
| monoethyl ether | | | |
| (DGME) | | | |
| Isopropanol (IPA) | 33% | 0.990 | 47.15 |
| Oleyl alcohol | 0 | 0 | |
| Polysorbate 80 | 0 | 0 | |
| Methyl salicylate | 0 | 0 | |
| Menthol | 0 | 0 | |
| TOTAL | 100.0% | 3.000 ^{a} | |

^{a} Weight as shown or multiples of thereof keeping Vial 1 and Vial 2 contents in the same ratios for a total weights of 6.0, 9.0, 12.0, 15.0 g, etc.

Table 25A and Table 25B provide examples of additional possible lemuteporfin formulations for adding active photosensitizer in a two-component system. The formulations were prepared as described above.

TABLE 25A

| Formulation | P1 | | P2 | | P3 | | P4 | | P5 | | P6 | | P7 | |
|---|---|---|---|---|---|---|---|---|---|---|---|---|---|---|
| Benzyl | (10 g) | 11.5% | (10 g) | 11.9% | (10 g) | 12% | (10 g) | 12.8% | (10 g) | 12.8% | (10 g) | 14.3% | (15 g) | 23.1% |
| alcohol | | | | | | | | | | | | | | |
| Isopropyl | (48.9 g) | 56.4% | (48.9 g) | 58.0% | (48.9 g) | 58.6% | (48.9 g) | 62.4% | (48.9 g) | 62.8% | (50 g) | 71.4% | (50 g) | 76.9% |
| alcohol, USP | | | | | | | | | | | | | | |
| (IPA) | | | | | | | | | | | | | | |
| Diethylene | (19 g) | 21.9% | (19 g) | 22.6% | (19 g) | 22.8% | (19 g) | 24.2% | (19 g) | 24.4% | (10 g) | 14.3% | 0 | |
| glycol | | | | | | | | | | | | | | |
| monoethyl | | | | | | | | | | | | | | |
| ether, NF | | | | | | | | | | | | | | |
| Oleyl | (5 g) | 5.8% | (5 g) | 5.9% | (5 g) | 6% | 0 | | 0 | | 0 | | 0 | |
| alcohol, | | | | | | | | | | | | | | |
| NF | | | | | | | | | | | | | | |
| Menthol, | (2.5) | 2.9% | (0.05 g) | 0.06% | 0 | | 0 | | 0 | | 0 | | 0 | |
| USP | | | | | | | | | | | | | | |
| Methyl | (0.8 g) | 0.9% | (0.8 g) | 0.9% | 0 | | 0 | | 0 | | 0 | | 0 | |
| salicylate, | | | | | | | | | | | | | | |
| NF | | | | | | | | | | | | | | |
| Polysorbate | (0.5 g) | 0.6% | (0.5 g) | 0.6% | (0.5 g) | 0.6% | (0.5 g) | 0.6% | 0 | | 0 | | 0 | |
| 80, NF | | | | | | | | | | | | | | |
| Total | (86.7 g) | 100% | (84.25 g) | 100% | (83.4 g) | 100% | (78.4 g) | 100% | (77.9 g) | 100% | (70 g) | 100% | (65 g) | 100% |

| | | | | | | | |
|---|---|---|---|---|---|---|---|
| Lemute- | 0.27 | 0.27 | 0.25 | 0.27 | 0.26 | 0.17 | 0.16 |
| porfin | | | | | | | |
| Solubility | | | | | | | |
| (mg/ml | | | | | | | |
| 25° C. | | | | | | | |
| at 24 h) | | | | | | | |

TABLE 25B

| Formulation | P8 | | P9 | | P10 | | P11 | | P12 | | P13 | | P14 | |
|---|---|---|---|---|---|---|---|---|---|---|---|---|---|---|
| Benzyl | (30 g) | 33.3% | (30 g) | 37.5% | (40 g) | 44.4% | (39 g) | 48.8% | (10 g) | 40.0% | (12)g | 50.0% | (10 g) | 50.0% |
| alcohol | | | | | | | | | | | | | | |
| Isopropyl | (50 g) | 55.6% | (50 g) | 62.5% | (40 g) | 44.4% | (41 g) | 51.3% | (10 g) | 40.0% | (10 g) | 41.7% | (5 g) | 25.0% |
| alcohol, USP | | | | | | | | | | | | | | |
| (IPA) | | | | | | | | | | | | | | |
| Diethylene | (10 g) | 11.1% | 0 | | (10 g) | 11.1% | 0 | | (5 g) | 20.0% | (2 g) | 8.3% | (5 g) | 25.0% |
| glycol | | | | | | | | | | | | | | |
| monoethyl | | | | | | | | | | | | | | |
| ether, NF | | | | | | | | | | | | | | |
| Oleyl | 0 | | 0 | | 0 | | 0 | | 0 | | 0 | | 0 | |
| alcohol, | | | | | | | | | | | | | | |
| NF | | | | | | | | | | | | | | |
| Menthol, | 0 | | 0 | | 0 | | 0 | | 0 | | 0 | | 0 | |
| USP | | | | | | | | | | | | | | |
| Methyl | 0 | | 0 | | 0 | | 0 | | 0 | | 0 | | 0 | |
| salicylate, | | | | | | | | | | | | | | |
| NF | | | | | | | | | | | | | | |
| Polysorbate | 0 | | 0 | | 0 | | 0 | | 0 | | 0 | | 0 | |
| 80, NF | | | | | | | | | | | | | | |
| Total | (90 g) | 100% | (80 g) | 100% | (90 g) | 100% | (80 g) | 100% | (25 g) | 100% | (24 g) | 100% | (20 g) | 100% |

| | | | | | | | |
|---|---|---|---|---|---|---|---|
| Lemute- | 0.49 | 0.43 | 0.93 | 0.84 | 0.96 | 1.14 | 1.66 |
| porfin | | | | | | | |
| Solubility | | | | | | | |
| (mg/ml | | | | | | | |
| 25° C. | | | | | | | |
| at 24 h) | | | | | | | |

The lemuteporfin equilibrium solubility at ambient room temperature in the final solution (mg/mL), and supersaturation at t=0 (not corrected for specific gravity), is shown in Table 26.

TABLE 26
Formulation Solubility

| Formulation | Equilibrium Solubility | Supersaturation |
|---|---|---|
| Batch I (P2) | 0.27 mg/mL | 443% |
| Batch J (P3) | 0.25 mg/mL | 484% |
| Batch K (P5) | 0.26 mg/mL | 498% |
| Batch M (P12) | 0.96 mg/mL | 415% |
| Batch N (P14) | 1.66 mg/mL | 301% |
| Batch P (P16) | 0.98 mg/mL | 362% |
| Batch Q (P17) | 1.18 mg/mL | 318% |
| Batch S (F20) | 1.136 mg/mL | 312% |
| Batch T and U (F21) | 1.246 mg/mL | 241% |

### Example 8

### Lemuteporfin Drug Localization in Human Sebaceous Glands Comparison of LTS (0.02%), LTS (0.1%) and LTO (0.2%)

Lemuteporfin sebaceous gland localization with the LTS formulation was studied in a human clinical study. The work was performed to evaluate two strengths (0.02%, 0.1%) of lemuteporfin topical solution (LTS) formulation for their potential to support the distribution of lemuteporfin-related fluorescence into sebaceous glands of the upper back of healthy subjects, either with or without prior skin preparation. An earlier generation formulation, lemuteporfin Topical Ointment (LTO) 0.2% under occlusion in combination with infrared (IR) heat skin preparation was tested in parallel as a control treatment because its sebaceous gland delivery properties had been previously studied. The composition of LTO had been determined to be non-optimal for delivery of lemuteporfin into human sebaceous glands. The safety and local tolerability of LTS, in combination with and without different skin preparation methods, was also evaluated in this study.

### Study Design

A partial-blind, sequential, randomized drug-localization study consisting of two cohorts of 10 healthy human subjects each (20 subjects total) was carried out under informed consent. Each of the 20 study subjects attended all scheduled visits and completed the study. The mean age of subjects was 24 years (range: 18-30 years). Eleven (55%) of the subjects were female. Cohorts 1 and 2 evaluated two different dose strengths of LTS, 0.02% w/w and 0.1% w/w, respectively. Each subject had four test sites (2 cm×2 cm) positioned on the upper back. Subjects received each of the four treatment regimens:
LTS without any skin preparation
LTS after skin preparation with micro-derm-abrasion (MDA)
LTS after skin preparation with dry heat from an IR heat device
LTO with plastic film occlusion after skin preparation with dry heat from IR device
   Each formulation was allowed to remain in contact with the skin for approximately 60 minutes. Upon completion of the contact time, excess material was removed from the test sites using clean gauze dampened with lukewarm water and then a 4 mm punch biopsy was taken from each test site.

### Sebaceous Gland Fluorescence Analysis

Biopsies were placed in Neg-50™ frozen section embedding medium and snap-frozen in liquid nitrogen. Samples were stored at −70° C. and shipped on dry ice to a histology laboratory with extensive experience in the required methodology. Tissue blocks were placed onto a chuck of a Microm EM500 Cryostat and then trimmed to expose the tissue area. Eight micron thick sections were cut onto microscope slides, which were immediately covered with a glass cover slip adhered by Prolong® Antifade (Molecular Probes) and stored in a light-opaque box at 4° C.

For each biopsy sample, approximately twenty slide sets were prepared. Each of these sets consisted of 3 slides. The first three sets were appraised for the absence/presence of sebaceous glands. Generally, the next five sets were omitted and the following three sets were assessed for the presence of sebaceous gland structures. This selection process continued until a total of nine sets with acceptable sebaceous gland presence were identified. However, if the last slide set had been evaluated without nomination of nine sets with adequate sebaceous gland representation, omitted sets were then examined in the sequence that they were prepared until nine sets were acquired. If nine sets were not obtainable from the biopsy sample, then the maximum available number of sets was ultimately evaluated.

Fluorescence microscopy was used to evaluate the distribution of lemuteporfin in the skin and to determine if there was specific accumulation of lemuteporfin in the sebaceous glands. Slides were viewed with a Zeiss Axiovert TV100 microscope equipped with a monochromatic Photometrics 350 camera (Roper Scientific). The sections were initially viewed under bright field illumination to identify sections with sebaceous glands. Images were then taken with epi-fluorescence illumination appropriate for lemuteporfin (excitation 425 nm; emission 690 nm). The exposure for each fluorescent image was 5 seconds with a 5× lens objective covering a 2×2 mm area at this magnification. Each image was taken at 16-bit depth which resulted in 65500 shades of grey. This setting gave increased precision for fluorescence detection. The display range (i.e. contrast intensity) for all samples was set to a scale of 500-5000 using Image-Pro Plus software. In previous studies, it was consistently observed that skin biopsy samples obtained from lemuteporfin-naïve skin exhibited no detectable fluorescence.

Biopsy sample images were appraised for the distribution of fluorescence within sebaceous glands examined by a panel of experienced evaluators who are blinded to the identity and the origin of the samples. With group consensus, samples were deemed positive for sebaceous gland uptake of lemuteporfin if the fluorescence distinctly revealed general gland structure and/or outlined gland lobules with greater intensity than the surrounding tissues. The non-parametric Chi-(X²)-square test was performed to reveal whether the observed differences in sebaceous gland lemuteporfin fluorescence results for the different treatments within each cohort were statistically significant.

### Results

In this drug-distribution study, the different skin preparation methods employed as well as application of the LTS/LTO formulations were generally well-tolerated. Edema was not observed at any test site. When localized skin erythema was observed, it was primarily associated with the skin preparation procedures.

The sebaceous gland localization of lemuteporfin applied in different topical formulations was assessed using tissue fluorescence image analyses. Lemuteporfin fluorescence signal was evident within hair follicles and sebaceous glands with the different test regimens although to various degrees. For all samples, there was no appreciable fluorescence signal in surrounding non-pilosebaceous structures. In some samples, strong lemuteporfin fluorescence was associated with plugs within the outer pore region of hair follicles. This circumstance produced a fluorescence flaring phenomenon that emanated into adjacent portions of these samples. Such observations were typically recorded as a negative result unless sufficiently prominent and separated sebaceous gland fluorescence was also present. Several sections exhibited drug fluorescence in the stratum corneum layer, suggesting that some residual drug remained on the skin surface.

For sections obtained from control (IR heat pretreatment plus 0.2% LTO with occlusion) sites, skin areas exposed to LTS at 0.1% combined with MDA or lower strength LTS (0.02%) with different pretreatments, approximately 20% of these slides had fluorescence signal evident within sebaceous glands (Table 27). Fluorescence image findings for the control sites (IR heat pretreatment plus 0.2% LTO) were similar for Cohorts 1 and 2 (19.2% and 19.1%, respectively) indicating the reproducibility of the treatment and analysis methodology. For subjects treated with LTS at 0.02% versus LTO at 0.2% under occlusion, there was no significant difference in the proportion of group samples with lemuteporfin-related sebaceous gland fluorescence as determined by the non-parametric Chi-square statistical test (X² value=1.36, 3 degrees of freedom, P=0.715).

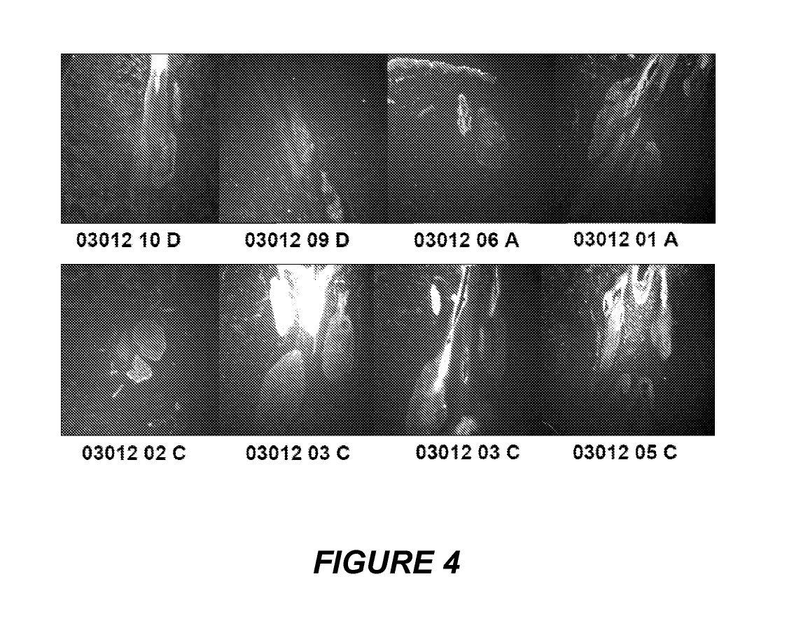

The test group with the highest number of positive biopsies, as defined as a biopsy sample with at least two fluorescence-positive slides sets from all sets evaluated, was Cohort 2 (0.1% LTS). For LTS at 0.1%, 6 of 9 of evaluable (sebaceous gland-containing) biopsies were deemed positive for sebaceous gland fluorescence (see FIG. 4 for fluorescence images of sebaceous glands). For the group that received IR heat treatment plus LTS at 0.1%, 7 of 9 evaluable biopsies were judged to be positive for drug-specific sebaceous gland fluorescence. For subjects treated with LTS at 0.1% in comparison to LTO at 0.2% under occlusion, there was a significant difference in the proportion of group samples exhibiting sebaceous gland lemuteporfin-specific fluorescence as determined by Chi-square statistical analysis (X² value=15, 3 degrees of freedom, P=0.002). Overall, subjects treated with LTS 0.1%, either alone or with IR heat pre-treatment, exhibited a greater extent of back skin sebaceous gland fluorescence than when MDA plus LTS at 0.1% or LTO 0.2% under occlusion following IR heat treatment was performed.

These data support the following conclusions. LTS enables distribution of lemuteporfin to the human sebaceous gland, as evidenced by the fact that in subjects administered LTS, lemuteporfin was observed in ≧50-70% of biopsies and 17-45% of biopsy slides via fluorescence microscopy. LTS enables improved distribution of lemuteporfin to the sebaceous gland relative to LTO, as evidenced by the fact that biopsy samples and slides were more frequently positive in subjects administered LTS than LTO under similar conditions (notwithstanding the fact that the concentration of lemuteporfin was 2 to 10-fold lower in LTS than in LTO). Higher concentrations of LTS enable better distribution to the sebaceous gland, as evidenced by the fact that biopsy samples and slides were more frequently positive in subjects administered 0.1% than 0.02% LTS. “Preparing” the skin by administering heat or microdermabrasion prior to applying LTS may not necessarily improve lemuteporfin distribution to the sebaceous gland, as evidenced by the fact that the frequency of positive biopsy samples and slides was not significantly higher in subjects who received such skin preparation procedures than in subjects who did not.

TABLE 27
Fluorescent Image Analysis Results

| | Cohort 1 (LTS 0.02%) | | | | Cohort 1 (LTS 0.1%) | | | |
|---|---|---|---|---|---|---|---|---|
| | | | | 0.2% | | | | 0.2% |
| | | LTS + | LTS + | LTO + | | LTS + | LTS + | LTO + |
| | LTS | MDA | Heat | Heat | LTS | MDA | Heat | Heat |
| Number of positive bi- | 5 | 4 | 6 | 4 | 6^{a} | 5 | 7^{b} | 3 |
| opsies per group of 10 | | | | | | | | |
| Fluorescent positive | 17.3 | 18.3 | 24.3 | 19.2 | 41.3 | 22.4 | 44.8 | 19.1 |
| slides (% slides) | | | | | | | | |

^{a}Excludes 2 negative biopsies, each with 1 slide showing a strong fluorescent signal in sebaceous glands
^{b}Excludes 1 biopsy with no sebaceous gland, and 1 biopsy with only 3 slides with sebaceous gland structure
MDA: Microderabrasion

### Example 9

### Determination of Sebum Excretion Ratio (SER) on a Subject's Forehead

A sebum excretion ratio may be used to monitor the efficacy of treatment of a subject, and may be determined as follows. The subject's forehead is first degreased, by doing the following: 1) moistening a cosmetic pad with water; 2) applying shampoo to the pad (an amount about the size of a quarter) and folding the pad in half to distribute the shampoo; 3) washing the subject's forehead gently using small circular motions, moving from the middle of the forehead to the temple and repeating once on each side; 4) wiping the forehead gently with water-moistened gauze; 5) patting the forehead dry with a clean cosmetic pad; 6) wiping the forehead with 70% isopropyl alcohol, working from the center of the forehead to the temple with three isopropyl alcohol pads for each side of the forehead, wiping the bottom half of the forehead with one pad, the top half with another, then unfolding a third pad and wiping the entire side of the forehead; and 7) letting the subject's forehead dry at least 5 minutes.

The SebuTape™ patch is carefully lifted from the carrier sheet and applied to the site, ensuring that the tape is applied smoothly to the skin surface with no wrinkles. The patch is firmly press to ensure that the tape is in good contact with the surface of the skin. After 30 min to 120 min (depending on the protocol), the patch is removed and transferred to the black rectangles on the storage card. The correct date, time and side the patch was applied to (i.e., left or right) is recorded in the comment section below the patch.

The storage card is scanned immediately after sampling with an image resolution of 600 dpi. Each image file is saved in a JPEG format into the appropriate folder using a descriptive filename. Using appropriate software such as PhotoShop® (Adobe, San Jose, Calif.), all the dark pixels on the patch are selected. The sebum output is represented by the black pixels which can then be converted to the sebum excretion rate by multiplying by a factor of 807.5.

### Example 10

### Stability of Lemuteporfin Supersaturated Solutions

### A. Stability of LTS Formulation for Vial 1 Benzyl Alcohol Solvent

Three formulations (Batches C (Table 9), D (Table 10) and H (Table 11)) in which the Vial 1 photosensitizer component consisted of benzyl alcohol and lemuteporfin at three lemuteporfin concentrations, 0.1, 0.075 and 0.05% w/w in the final combined LTS solution were examined for stability after reconstitution with the remaining excipients in Vial 2.

Vial 2 contents were added to Vial 1 for each formulation, mixed and sampled at time 0 and 4 hours after reconstitution. The samples were filtered through a 0.22 μm filter before analysis by HPLC. This analysis was performed to ensure that the combined product had adequate stability and would not precipitate before administration to a subject. The data are presented in Table 28.

TABLE 28
LTS Batches C, D and H Reconstituted Solution

| | | Target | Lemuteporfin |
|---|---|---|---|
| Samples | Time | % w/w | % w/w |
| C-0.1% w/w Mixed and filtered | 0 | 0.1 | 0.1064 |
| C-0.1% w/w Mixed and filtered | 4 hr. | 0.1 | 0.1065 |
| D-0.075% w/w Mixed and filtered | 0 | 0.075 | 0.0765 |
| D-0.075% w/w Mixed and filtered | 4 hr. | 0.075 | 0.0769 |
| H-0.05% w/w Mixed and filtered | 0 | 0.05 | 0.0503 |
| H-0.05% w/w Mixed and filtered | 4 hr. | 0.05 | 0.0504 |

The reconstitution data demonstrated that at 4 hours and up to 48 hours post-reconstitution, lemuteporfin was still dissolved and had not precipitated out of the LTS solution for the formulations tested.

### B. Stability of LTS Formulation for Vial 1 Benzyl Alcohol and DGME Solvent

Two formulations were examined in which the photosensitizing component in Vial 1 consisted of DGME, benzyl alcohol and lemuteporfin at two lemuteporfin concentrations of 0.1 (Batch A, Table 7) and 0.075% (Batch B, Table 8) in the final formulation. Vial 2 contents were added to Vial 1 contents, mixed and sampled at 0 and 4 hours after reconstitution. The samples were filtered through 0.2 μm filter before analysis. This experiment was performed to ensure that the combined product has adequate stability and would not precipitate before administration to a subject. The data obtained are presented in Table 29.

TABLE 29
LTS Batches A and B Reconstituted Solution

| | Target % | Lemuteporfin |
|---|---|---|
| Samples | w/w | % w/w |
| A-0.1% w/w Mixed and filtered | 0.1 | 0.099 |
| time 0 | | |
| A-0.1% w/w Mixed and filtered | 0.1 | 0.100 |
| time 4 hours | | |
| B-0.075% w/w Mixed and filtered | 0.075 | 0.076 |
| time 0 | | |
| B-0.075% w/w Mixed and filtered | 0.075 | 0.077 |
| time 4 hours | | |

The reconstitution data demonstrate that up to 4 hours, lemuteporfin was still dissolved and had not precipitated out of solution. We have also found that the chemical stability of the lemuteporfin in Vial 1 of Batch C extends to at least twelve months at 5° C., and at least 6 months at 40° C. Additional studies demonstrated that lemuteporfin TK1 was physically stable for at least 48 hours.

### C. Physical Stability of 0.1% LTS (Visual Assessment)

Using Batch C from Table 9, 0.9 g of the 1% lemuteporfin solution in benzyl alcohol was combined with the entire contents of an inactive vial to yield a 0.1% LTS. This solution demonstrated acceptable physical stability by visual assessment at every time point. The results are shown in Table 30.

TABLE 30
Physical Stability of LTS

| | (w/w)% active concentration | | | | | | | |
|---|---|---|---|---|---|---|---|---|
| | 2 hr. | 4 hr. | 6 hr. | 16 hr. | 24 hr. | 48 hr. | 72 hr. | 1 Week |
| 0.9-g 1% in | Sol- | Sol- | Sol- | Sol- | Sol- | Sol- | Sol- | Sol- |
| benzyl | uble | uble | uble | uble | uble | uble | uble | uble |
| alcohol + | | | | | | | | |
| inactive vial | | | | | | | | |

### D. Short Term Physical Stability of 0.1% LTS as in Table 9 (Batch C) (HPLC Assessment Over 48 Hours)

Using Batch C from Table 9, constituted 0.1% (w/w) lemuteporfin solutions were filtered and sampled at various time points (0, 2 h, 4 h, 6 h, 8 h, 10 h, 16 h, 24 h and 48 h) and assayed for lemuteporfin content. The results presented in Table 31 show that the content of lemuteporfin in 0.1% LTS after filtration is stable and remains at about 100% of formula strength for at least 48 hours.

TABLE 31
LTS Short Term Physical Stability

| Time Point | % Formula |
|---|---|
| 0 hr | 98.98 |
| 2 hr | 98.91 |
| 4 hr | 99.21 |
| 6 hr | 99.09 |
| 8 hr | 99.12 |
| 10 hr | 99.12 |
| 16 hr | 99.99 |
| 24 hr | 98.98 |
| 48 hr | 98.91 |

### E. Short Term Chemical and Physical Stability of 0.3% LTS (HPLC Assessment Over 48 Hours)

Using Batch U (F21) from Table 23, constituted LTS (0.3%) solutions were filtered and sampled at various time points (0, 2 h, 4 h, 6 h, 8 h, 10 h, 16 h, 24 h and 48 h) and assayed for lemuteporfin content and Total Related Compounds (TRC). Results showed that the content of lemuteporfin in 0.3% LTS is stable after filtration and remained so throughout 48 hours. The assay showed no differences between the filtered and unfiltered solutions, demonstrating that the supersaturated solution is physically stable over 48 hours (Tables 32 and 33).

TABLE 32
Unfiltered Solutions

| Time | | | | | % | | |
|---|---|---|---|---|---|---|---|
| Point | | Sample | Area | | Formula | Area % | Area % |
| (h) | Replicate | weight (g) | Lemuteporfin | % w/w | (0.3) | Lemuteporfin | TRC^{§} |
| 0 | 1 | 0.9066 | 9,537,157 | 0.2931 | 97.7 | 98.98 | 1.02 |
| | 2 | 0.8996 | 9,482,591 | 0.2937 | | 99.08 | 0.92 |
| | 3 | 0.8996 | 9,451,525 | 0.2927 | | 99.01 | 0.99 |
| | | | Ave | 0.2932 | | 99.02 | 0.98 |
| | | | RSD | 0% | | 0% | 5% |
| 4 h | 1 | 0.9058 | 9,655,550 | 0.2970 | 97.7 | 99.03 | 0.97 |
| | 2 | 0.9116 | 9,519,734 | 0.2910 | | 98.83 | 1.17 |
| | 3 | 0.9057 | 9,457,584 | 0.2910 | | 99.11 | 0.89 |
| | | | Ave | 0.2930 | | 98.99 | 1.01 |
| | | | RSD | 1% | | 0% | 14% |
| 8 h | 1 | 0.9099 | 9,381,219 | 0.2873 | 96.3 | 99.01 | 0.99 |
| | 2 | 0.8993 | 9,325,983 | 0.2890 | | 98.95 | 1.05 |
| | 3 | 0.9016 | 9,397,767 | 0.2904 | | 99.5 | 0.5 |
| | | | Ave | 0.2889 | | 99.15 | 0.85 |
| | | | RSD | 1% | | 0% | 36% |
| 24 h | 1 | 0.9043 | 9,464,714 | 0.2916 | 97.4 | 99.12 | 0.88 |
| | 2 | 0.9141 | 9,608,013 | 0.2929 | | 98.99 | 1.01 |
| | 3 | 0.9063 | 9,509,647 | 0.2924 | | 98.72 | 1.28 |
| | | | Ave | 0.2923 | | 98.94 | 1.06 |
| | | | RSD | 0% | | 0% | 19% |
| 48 h | 1 | 0.9101 | 9,455,169 | 0.2895 | 96.4 | 98.95 | 1.05 |
| | 2 | 0.9000 | 9,342,987 | 0.2893 | | 98.96 | 1.04 |
| | 3 | 0.9050 | 9,374,680 | 0.2886 | | 98.91 | 1.09 |

| | | | |
|---|---|---|---|
| Ave | 0.2891 | 98.94 | 1.06 |
| RSD | 0% | 0% | 2% |

^{§}TRC, Total Related Compounds

TABLE 33
Filtered Solutions

| Time | | | | | % | | |
|---|---|---|---|---|---|---|---|
| Point | | Sample | Area | | Formula | Area % | Area % |
| (h) | Replicate | weight (g) | Lemuteporfin | % w/w | (0.3) | Lemuteporfin | TRC^{§} |
| 0 | 1 | 0.9138 | 9,519,009 | 0.2903 | 97.4 | 99.03 | 0.97 |
| | 2 | 0.9053 | 9,595,378 | 0.2953 | | 98.95 | 1.05 |
| | 3 | 0.9165 | 9,584,788 | 0.2914 | | 99.02 | 0.98 |
| | | | Ave | 0.2923 | | 99.00 | 1.00 |
| | | | RSD | 1% | | 0% | 4% |
| 4 h | 1 | 0.9047 | 9,421,886 | 0.2902 | 96.7 | 99.04 | 0.96 |
| | 2 | 0.9110 | 9,429,024 | 0.2884 | | 99.02 | 0.98 |
| | 3 | 0.9124 | 9,539,854 | 0.2913 | | 98.9 | 1.1 |
| | | | Ave | 0.2900 | | 98.99 | 1.01 |
| | | | RSD | 1% | | 0% | 7% |
| 8 h | 1 | 0.9057 | 9,370,453 | 0.2883 | 96.5 | 99.07 | 0.93 |
| | 2 | 0.9005 | 9,404,665 | 0.2910 | | 99.07 | 0.93 |
| | 3 | 0.9022 | 9,362,953 | 0.2892 | | 98.97 | 1.03 |
| | | | Ave | 0.2895 | | 99.04 | 0.96 |
| | | | RSD | 0% | | 0% | 6% |
| 24 h | 1 | 0.9051 | 9,443,131 | 0.2907 | 96.9 | 99.07 | 0.93 |
| | 2 | 0.9122 | 9,528,965 | 0.2911 | | 98.85 | 1.15 |
| | 3 | 0.9009 | 9,380,970 | 0.2901 | | 98.96 | 1.04 |
| | | | Ave | 0.2906 | | 98.96 | 1.04 |
| | | | RSD | 0% | | 0% | 11% |
| 48 h | 1 | 0.9083 | 9,638,435 | 0.2957 | 99.6 | 98.77 | 1.23 |
| | 2 | 0.9050 | 9,781,505 | 0.3012 | | 98.98 | 1.02 |
| | 3 | 0.9026 | 9,701,269 | 0.2995 | | 98.84 | 1.16 |

| | | | |
|---|---|---|---|
| Ave | 0.2988 | 98.86 | 1.14 |
| RSD | 1% | 0% | 9% |

^{§}TRC, Total Related Compounds

### Example 11

### PDT Treatment of Acne Human Subjects Using Lemuteporfin PDT

This is Phase I/II, double-blind, sequential study of the effect of photodynamic therapy (PDT) with Lemuteporfin Topical Solution (LTS) in healthy subjects and in subjects with mild acne. A maximum of 202 subjects will be enrolled in 4 stages (12 in Stage 1, 30 to 90 in stage 2, 30 to 60 in Stage 3, and 20 to 40 in Stage 4). The study will be performed according to FDA guidelines and subjects will be asked to give informed consent.

### Stage 1

Twelve (12) healthy subjects will be assigned to two different cohorts in Stage 1. This stage will evaluate LTS-PDT with six light doses (25, 50, 75, 125, 225, and 300 J/cm²; 688 nm) to determine the maximum tolerated dose of red light (MTD_{red}). Light exposure will take place 60±5 minutes after LTS application. The MTD_{red} is defined as the light dose that either: (1) results in the highest tolerable blanchable erythema and/or discomfort associated with light treatment or (2) is the highest light dose tested with no intolerable discomfort or erythema. The six light treatments will be administered across two cohorts. Each cohort will include six subjects. There will be four test sites of 5 cm×8 cm each on the upper back of each subject.

On the same day, the following will be applied to each of the four test sites:

Cohort 1:
No treatment (negative control)
LTS 0.1% application followed by a light dose of 25 J/cm² (50 mW/cm²)
LTS 0.1% application followed by a light dose of 50 J/cm² (50 mW/cm²)
LTS 0.1% application followed by a light dose of 75 J/cm² (50 mW/cm²)

Cohort 2:
No treatment (negative control)
L TS 0.1% application followed by a light dose of 125 J/cm² (80 mW/cm²)
L TS 0.1% application followed by a light dose of 225 J/cm² (80 mW/cm²)
L TS 0.1% application followed by a light dose of 300 J/cm² (80 mW/cm²)
   If three or more subjects experience intolerable discomfort (grade 4 on a 0-4-point scale) at a light dose, that dose will be discontinued.

At each study visit (Day 0, 1 and 14), erythema and edema will be evaluated through a scoring system and assessment of hyperpigmentation and papulopustular acneiform reaction will be performed, along with adverse events evaluation. Subjects will also be interviewed immediately after red light treatment to assess discomfort. Clinical laboratory tests and vital signs assessment will also be part of the safety evaluations. Subjects will be followed for 2 weeks after the treatment.

### Stage 2

Approximately 30 subjects with at least two inflammatory lesions of acne on the forehead will be assigned to three different cohorts in Stage 2, with the possibility to add up to a maximum of six additional cohorts of ten subjects. The overall objective will be to assess safety and to measure clinical activity via sebum excretion rates and biopsies after escalating light doses up to the MTD_{red} on the face of subjects with acne lesions. This will be assessed by treating ten subjects in each cohort with LTS 0.1% and vehicle applications to the forehead followed by exposure to red light. Administration of active treatment and vehicle will be split to one half of the forehead, separated by the midline. Determination of the application of the active treatment and vehicle will be randomized and performed in a double-blind fashion. The maximum red light dose that may be administered will be the MTD_{red} dose determined in Stage 1.

Light dose and LTS/vehicle application will be done as follows for each cohort:
Cohort 1: LTS 0.1% and vehicle application (60±5 minute incubation) followed by a light dose of 25 J/cm² (50 mW/cm²)
Cohort 2: LTS 0.1% and vehicle application (60±5 minute incubation) followed by a light dose of 75 J/cm² (50 mW/cm²)
Cohort 3: LTS 0.1% and vehicle application (60±5 minute incubation) followed by a light dose of 150 J/cm2 (50 mW/cm²)
   After review of efficacy and safety data from Cohorts 1 to 3 by the sponsor and the investigator, a decision will be made about adding additional cohorts in order to enhance efficacy or improve tolerability. A maximum of 6 cohorts of 10 subjects may be added. The time of incubation with LTS and vehicle as well as the red light dose used for subjects in these additional cohorts will be determined based upon results from Cohorts 1 to 3. The parameters used with subjects in the additional cohorts will not exceed parameters tested in Stage 1. Specifically, the maximum light dose used for subjects in the additional cohorts will be the MTD_{red}. The irradiance used for subjects in the additional cohorts will be one of the two irradiances tested in Stage 1 (50 mW/cm² or 80 mW/cm²). The incubation time between LTS application and red light exposure for subjects in the additional cohorts will not exceed 60 minutes.

Acne lesion count and Sebum Excretion Rate (SER) using the Sebutape® will be monitored at screening, Day 0, 1 and 14 on all subjects in Stage 2. In addition, 50% of subjects in cohorts 1 to 3 will have one 3 mm punch biopsy sample taken at the forehead from the vehicle and LTS-treated regions 24±4 hours after LTS-PDT treatment for further assessments. These samples will subsequently be processed and tissue sections will be evaluated for evidence of PDT action using immunohistochemical methods.

At each study visit (Day 0, 1 and 14), erythema and edema will be evaluated through a scoring system and assessment of hyperpigmentation and papulopustular acneiform reaction will be performed, along with adverse events evaluation. Subjects will also be interviewed immediately after red light treatment to assess discomfort. Clinical laboratory tests and vital signs assessment will also be part of the safety evaluations. Subjects will be followed for 2 weeks after the treatment. For subjects consenting to photographs, high quality digital medical photographs of the forehead will be taken at Day 0 prior to LTS/vehicle application, after light treatment on day 0, and at follow-up visits on Day 1, 7 and 14.

### Stage 3

Approximately 30 and up to 60 subjects with at least 2 inflammatory lesions of acne on the forehead will be assigned to 3 different cohorts in Stage 3. The overall objective will be to assess safety and to measure clinical activity via sebum excretion rates following LTS/vehicle-PDT treatment on the face of subjects with acne lesions. This will be assessed by treating 10 to 20 subjects in each cohort. LTS 0.1% will be applied to one side of the forehead and vehicle on the other side followed by red light exposure. Determination of the application of the active treatment and vehicle will be randomized and performed in a double-blind fashion. Determination of the red light dose used in Stage 3 will be made based data from subjects in Stage 2. The maximum red light dose will not exceed the MTDred achieved in Stage 1 (300 J/cm2).

Light dose and LTS/vehicle application will be done as follows for each cohort:
Cohort 1: Split LTS-PDT treatments performed on Day 0.
LTS 0.1% (split dose: 0.0375 mg/cm² for a total average dose of 1.5 mg prior to each light dose)
   Half of the total volume of LTS 0.1% and vehicle will be applied for 30±5 minutes followed by light exposure at 150 J/cm² (50 mW/cm²) with the possibility of up to 300 J/cm² (80 mW/cm²). Following this first treatment, the remaining volume of LTS 0.1%/vehicle will be applied for another 30±5 minutes followed by a second light exposure at 150 J/cm² (50 mW/cm²) up to 300 J/cm² (80 mW/cm²).
Cohort 2: One LTS-PDT treatment performed at Day 0 followed by another treatment at Day 3.
LTS 0.1% (dose: 0.075 mg/cm2 for a total average dose of 3 mg)
   LTS 0.1% and vehicle will be applied for 60±5 minutes followed by light exposure at 150 J/cm² (50 mW/cm²) with the possibility of up to 300 J/cm² (80 mW/cm²) on Day 0. The same treatment will be repeated at Day 3.
Cohort 3 and 3b: Split LTS-PDT treatments performed on Day 0 and repeated at Day 3.
LTS 0.1% (split dose: 0.0375 mg/cm² for a total average dose of 1.5 mg prior to each light dose)
   Half of the total volume of LTS 0.1% and vehicle will be applied for 30±5 minutes followed by light exposure at 150 J/cm² (50 mW/cm²) with the possibility of up to 300 J/cm2 (80 mW/cm²). Then, the remaining volume of LTS 0.1%/vehicle will be applied for another 30±5 minutes followed by a second light exposure at 150 J/cm² (50 mW/cm²) up to 300 J/cm² (80 mW/cm²). The same treatments will be repeated at Day 3.

Acne lesion count will be monitored at screening, Day 0, 7 and 14 on all subjects in Stage 3. Sebum Excretion Rate (SER) using the Sebutape® will be monitored at screening, pre-randomization, Days 0, 7, 8, 14 and 15 on all subjects in Stage 3 from Cohorts 1 to 3. For the subjects in Cohort 3b, the Sebutape will be used only at screening visit to determine subject's eligibility.

For subjects in Stage 3, who consent to photographs, high quality digital medical photographs of the forehead will be taken prior to LTS/vehicle application and after light treatment on Day 0 and Day 3 (if applicable), and at follow-up visits on Day 1, 4 (if applicable), 7 and 14. For the 10 additional subjects in Cohort 3b, photographs will be mandatory in order to measure skin fluorescence with the Visia at all study visits.

### Stage 4

Approximately 20 and up to 40 subjects with at least 2 inflammatory lesions of acne on the forehead will be assigned to 2 cohorts in Stage 4. The overall objective will be to assess the treatment effects and safety of LTS/vehicle-PDT (0.1% and 0.3%) treatment on the face. This will be assessed by treating 10 to 20 subjects in each cohort, using two formulations of LTS/vehicle. Subjects enrolled in Cohort 1 will receive LTS 0.1% applied to one side of the forehead and its matching vehicle on the other side followed by red light exposure. Safety data from subjects enrolled in Cohort 1 will be reviewed by the Investigator and Sponsor prior to proceeding with enrollment in Cohort 2. Cohort 2 will receive LTS 0.3% applied to one side of the forehead and its matching vehicle on the other side followed by red light exposure. Dosing for each cohort is outlined below. Determination of the application of the active treatment and vehicle will be randomized and performed in a double-blind fashion. The red light dose to be used in Stage 4 will be made based on data from subjects in Stages 2 and 3. The maximum red light dose will not exceed the MTDred achieved in Stage 1 (300 J/cm²).

Light dose and LTS/vehicle application will be done as follows for Cohorts 1 and 2:
Cohort 1: Split LTS/PDT treatments performed at Day 0, Wks 1, 2, 3, 4, 5, 6 and 7 LTS 0.1% (split dose: 0.075 mg/cm2 for a total average dose of 3 mg prior to each light dose)
   LTS and is matching vehicle will be applied and left for a contact time of 30±5 minutes followed by light exposure at 150 J/cm² (50 mW/cm²) with the possibility of up to 300 J/cm² (80 mW/cm²). Following this treatment, a second application of LTS and vehicle will be applied for 30±5 minutes followed by a second light exposure at the same light dose. The same split treatment will be repeated every week for a total of 8 treatments.
Cohort 2: Split LTS/PDT treatments performed at Day 0, Wks 1, 2, 3, 4, 5, 6 and 7 LTS 0.3% (split dose: 0.150 mg/cm² for a total average dose of 6 mg prior to each light dose)
   LTS and its matching vehicle will be applied and left for a contact time of 30±5 minutes followed by light exposure at 150 J/cm² (50 mW/cm²) with the possibility of up to 300 J/cm² (80 mW/cm²). Following this treatment, a second application of LTS and vehicle will be applied for 30±5 minutes followed by a second light exposure at the same light dose. The same treatments will be repeated every week for a total of 8 treatments.

Acne lesion counts will be done at screening, Day 0, and Week 3, 7 and 11 visits on all subjects in Stage 4. Sebum Excretion Rate (SER) using Sebutape® will be done at the screening visit to determine the subject's eligibility and at Week 3, 7 and 11 visits.

For subjects in Stage 4, high quality digital medical photographs of the forehead will be taken at Screening, prior to LTS/vehicle application and after light treatment on treatment days, and at the study exit visit at Week 11. The medical photographs will be also be used to measure skin fluorescence, an indirect measurement of sebum excretion, at all study visits.

### Statistical Analysis

### Sample Size

### Stage 1

There is no statistical rationale for the selected sample size for this stage. The sample size was selected based upon prior experience to ensure that the safety and tolerability will be adequately assessed while minimizing unnecessary exposure on healthy subjects.

### Stage 2

Based on paired Student's t-test, a sample size of 10 subjects in cohorts 1 to 3 will achieve a power of 80% to detect a difference from baseline at Day 14 of 1.42 in sebum excretion rate, assuming an average sebum excretion rate of 6.5 at baseline and no improvement in sebum excretion rate on the vehicle side. To control the test multiplicity, the significance level of 0.05 was adjusted using a Bonferroni correction (0.05/3=0.0167).

### Stage 3

Based on paired Student's t-test, a sample size of 10 subjects per cohort will achieve a power of 80% to detect a difference from baseline at Day 14 of 1.42 in sebum excretion rate assuming an average sebum excretion rate of 6.5 at baseline, no improvement in sebum excretion rate on the vehicle side. To control test multiplicity, the significance level of 0.05 was adjusted using with a Bonferroni correction (0.05/3=0.0167).

### Stage 4

The sample size for Stage 4 is not driven by a power analysis but rather by maintaining a consistent sample size with the other stages. Differences in measures of sebum excretion from baseline between vehicle and LTS treatment sides will be summarized using mean, standard deviation, median, minimum, and maximum determinations. A repeated measures ANOVA will be used to compare changes in sebum excretion rate for each method of measuring sebum excretion. Factors in the model will be treatment arm, subject and time. Additionally, univariate analyses will be conducted at each visit and interpreted to be statistically significant if the repeated measures “Tests of Hypotheses for Between Subjects Effect” p-value is significant. Lastly, the time by treatment interaction will provide information regarding the consistency of the treatment effect difference between treatments at the visit time points.

### Statistical and Analytical Plans

Continuous variables will be summarized in tables and will include the number of subjects, mean, standard deviation, median, min and max. Categorical variables will be presented in tables as frequencies and percentages. All statistical tests will be two-sided and will be performed with a significant level of 0.05. All subjects enrolled in the study who received LTS-PDT treatment will be included in the analyses. The safety population will be defined as all subjects who received the LTS-PDT treatment.

### Analysis of Demographic and Baseline Data

Demographic and baseline data will be analyzed for all enrolled subjects who received LTS-PDT treatment. Subject demographic and baseline characteristics will be summarized by mean, standard deviation, median, minimum, and maximum for continuous variables; and by counts and percentages for categorical variables. Summaries will be provided separately for each stage.

### Efficacy Analysis (Stages 2 and 3 Only)

Differences in SER values and skin fluorescence from baseline and between vehicle and LTS 0.1% treatment sides will be summarized using mean, standard deviation, median, minimum, and maximum determinations. An ANOVA for repeated measures will be used to compare changes in sebum excretion rate and skin fluorescence between the LTS and vehicle treated side. In addition, 95% confidence interval of the change in sebum excretion rate will be provided for each cohort.

### Efficacy Analysis (Stage 4)

Differences in measures of sebum excretion from baseline between vehicle and LTS treatment sides will be summarized using mean, standard deviation, median, minimum, and maximum determinations. A repeated measures ANOVA will be used to compare changes in sebum excretion rate for each method of measuring sebum excretion. Factors in the model will be treatment arm, subject and time. Additionally, univariate analyses will be conducted at each visit and interpreted to be statistically significant if the repeated measures “Tests of Hypotheses for Between Subjects Effect” p-value is significant. Lastly, the time by treatment interaction will provide information regarding the consistency of the treatment effect difference between treatments at the visit time points.

These data will be displayed graphically. A 95% confidence interval of the change in sebum excretion rate will be provided for each visit, cohort, treatment and method. The overall statistical evaluations of this section will be performed at a uniform alpha level of 0.05 to provide a metric for exploring the robustness of the various methods of evaluating the sebum excretion rate. No correction for controlling multiplicity will be performed. Correlation analyses will be performed to assess the consistency of the methods for measuring sebum excretion. Descriptive statistics for the change from baseline in total lesion counts will be presented for the visits by treatment group and cohort.

### Safety Analysis

### Stage 1

Discomfort scores will be summarized by test site (control and each light dose). Erythema scores, edema scores, presence or absence of PDT-induced papulopustular acneiform reaction, and presence or absence of hyperpigmentation will be summarized by test site, assessment visit and cohort. The MTDred will be summarized for each subject. AEs will be coded using MedDRA with the number and percentage of subjects experiencing an AE and the total number of AEs summarized by system organ class, preferred term, and test site (control and each light dose). Concomitant medications will be coded with the WHO-Drug Dictionary and listed by subject. Clinically significant chemistry and hematology laboratory values will be recorded as AEs. Vital signs will be listed by subject.

### Stages 2, 3 and 4

Discomfort scores will be summarized by cohort and treatment. Erythema scores, edema scores, presence or absence of PDT-induced papulopustular acneiform reaction, and presence or absence of hyperpigmentation will be summarized by treatment, assessment visit and cohort. AEs will be coded using MedDRA with the number and percentage of subjects experiencing an AE and the total number of events summarized by system organ class, preferred term, cohort and treatment. Concomitant medications will be coded with the WHO-DD and listed by subject. Clinically significant chemistry and hematology laboratory values will be recorded as AEs. Vital signs will be listed by subject.

### Treatment Assignment

Following completion of Stage 1 and Cohorts 1 through 3 in Stage 2, treatment assignments for each cohort will be released to the Sponsor after the last subject has completed the Day 14 visit. Treatment assignment information will support decisions about whether additional cohorts may be added to the study. Treatment assignment information will not be shared with study site staff.

### Example 12

### Sebum Diffusion Capillary Model for Formulation Screening

Formulations TK1, P12, P14, and P15 were prepared as described above and were screened for use in treating hyperactive sebaceous gland disorders using a sebum diffusion capillary model. First, glass capillaries were filled with synthetic sebum, as described in Lu et al., “Comparison of artificial sebum with human and hamster sebum samples”, Inter. Jour. of Pharm., 367 (2009) 37-43 (Sebum L). Filled capillaries (n=7) were dipped in a small amount of formulation (about 250 ul for 7 capillaries) and placed in a capped conical centrifuge tube. Care was taken to avoid evaporation of the test solution. At set diffusion time points, the capillaries were carefully blotted dry and the first 5 mm of each capillary was snapped off with a diamond knife. The content of seven of the 5-mm sections were dissolved in an organic solvent mixture and assayed for lemuteporfin by HPLC. The amount of lemuteporfin that diffused into the artificial sebum is reported as a function of contact time. Results for 60 minutes of diffusion time at 32.5° C. are shown in Table 34.

TABLE 34
Diffusion into Artificial Sebum

| Formula | μg lemuteporfin per ml sebum | μg lemuteporfin |
|---|---|---|
| TK1 | 3.5 | 0.49 |
| P14 | 8.0 | 1.12 |
| P12 | 11.3 | 1.58 |
| P15 | 9.8 | 1.37 |

### Example 13

### Sebum Diffusion Capillary Model Comparison of Batch U, Formulation F21 and Batch C, Formulation TK1

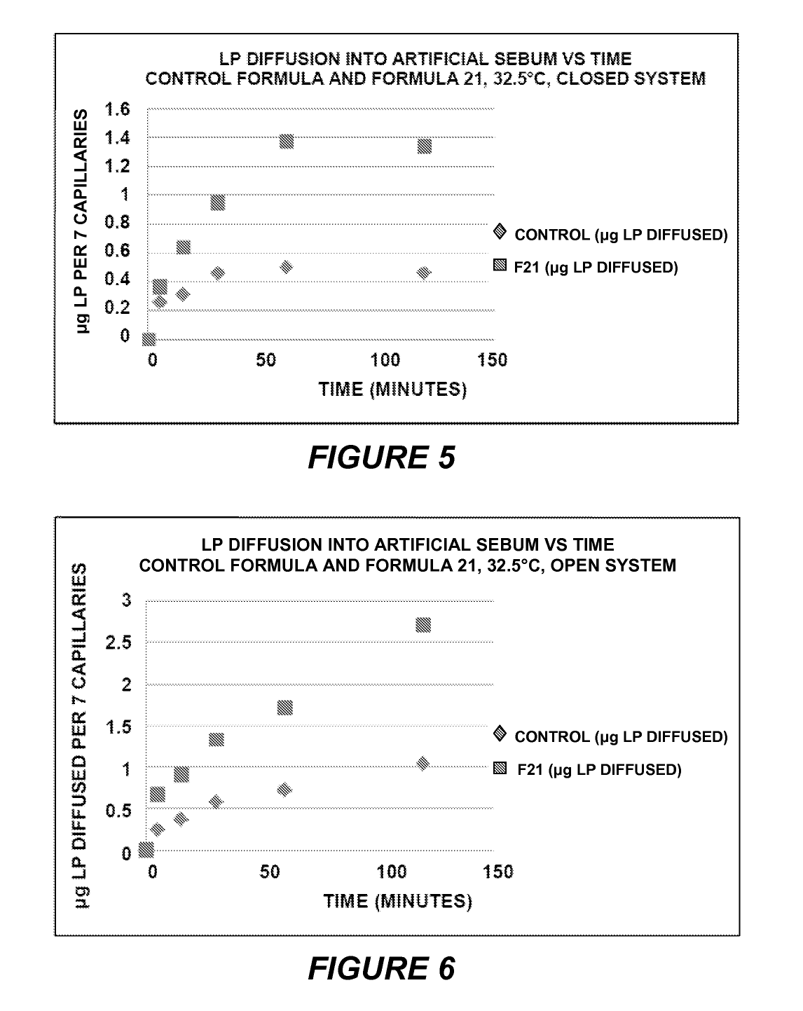

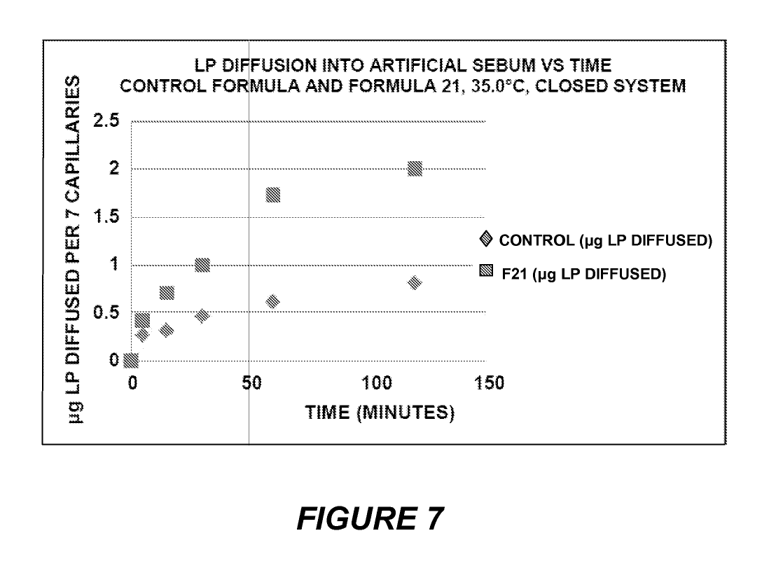

Glass capillary were filled with synthetic sebum, as described in Lu et al. (2009). Filled capillaries (n=7) were dipped in small amount of formulation (about 250 ul for 7 capillaries) and placed in a capped conical centrifuge tube. At set time points, the capillaries were carefully blotted dry and the first 5 mm of the tube was snapped off with a diamond knife. The contents of seven of the 5-mm sections were dissolved in an organic solvent mixture and assayed for lemuteporfin by HPLC. The amount of lemuteporfin that has diffused into sebum is reported as a function of contact time. Results comparing LTS, 0.3% (F21) to LTS, 0.1% (Formulation TK1 as shown for Batch C) are shown below for a closed system at 32.5° C. (Table 35), an open system at 32.5° C. (Table 36) and a closed system at 35° C. (Table 37). The results are shown graphically in FIGS. 5, 6 and 7 respectively.

TABLE 35
Summary of Lemuteporfin Diffusion in a Closed System at 32.5° C.

| | μg lemuteporfin Diffused | | | | |
|---|---|---|---|---|---|
| | per 7 Capillaries | | | | |
| | Replicate: | | | | |
| Test Condition | #1 | #2 | #3 | Mean | SD |
| 5 min Cont ^{a} 32.5° C. | 0.269 | 0.232 | 0.283 | 0.26 | 0.03 |
| 15 min Cont 32.5° C. | 0.293 | 0.348 | 0.302 | 0.31 | 0.03 |
| 30 min Cont 32.5° C. | 0.309 | 0.575 | 0.491 | 0.46 | 0.14 |
| 60 min Cont 32.5° C. | 0.622 | 0.428 | 0.449 | 0.50 | 0.11 |
| 120 min Cont 32.5° C. | 0.642 | 0.429 | 0.303 | 0.46 | 0.17 |
| 5 min F21 32.5° C. | 0.180 | 0.568 | 0.356 | 0.37 | 0.19 |
| 15 min F21 32.5° C. | 0.457 | 0.863 | 0.599 | 0.64 | 0.21 |
| 30 min F21 32.5° C. | 0.981 | 0.764 | 1.100 | 0.95 | 0.17 |
| 60 min F21 32.5° C. | 1.024 | 0.993 | 2.107 | 1.37 | 0.63 |
| 120 min F21 32.5° C. | 1.660 | 1.04 | 1.330 | 1.34 | 0.31 |

^{a} Cont = Control = LTS, 0.1% (formula TK1 as described for Batch C)

TABLE 36
Summary of Lemuteporfin diffusion at 32.5° C. in an
Open System

| | μg lemuteporfin Diffused | | | | |
|---|---|---|---|---|---|
| | per 7 Capillaries | | | | |
| | Replicate: | | | | |
| Test Condition | #1 | #2 | #3 | Mean | SD |
| 5 min Cont 32.5° C. | 0.324 | 0.151 | 0.261 | 0.25 | 0.09 |
| 15 min Cont 32.5° C. | 0.308 | 0.312 | 0.493 | 0.37 | 0.11 |
| 30 min Cont 32.5° C. | 0.787 | 0.400 | 0.535 | 0.57 | 0.20 |
| 60 min Cont 32.5° C. | 0.749 | 0.571 | 0.854 | 0.72 | 0.14 |
| 120 min Cont 32.5° C. | 0.747 | 0.918 | 1.446 | 1.04 | 0.36 |
| 5 min F21 32.5° C. | 0.644 | 0.688 | 0.697 | 0.68 | 0.03 |
| 15 min F21 32.5° C. | 1.196 | 0.815 | 0.696 | 0.90 | 0.26 |
| 30 min F21 32.5° C. | 2.239 | 0.722 | 1.042 | 1.33 | 0.80 |
| 60 min F21 32.5° C. | 1.634 | 1.355 | 2.139 | 1.71 | 0.40 |
| 120 min F21 32.5° C. | 2.961 | 2.527 | 2.619 | 2.70 | 0.23 |

^{a} Cont = Control = LTS, 0.1% (formula TK1 as described for Batch C)

TABLE 37
Summary of Lemuteporfin diffusion at 35.0° C. in a
Closed System

| | μg lemuteporfin Diffused | | | | |
|---|---|---|---|---|---|
| | per 7 Capillaries | | | | |
| | Replicate: | | | | |
| Test Condition | #1 | #2 | #3 | Mean | SD |
| 5 min Cont. 35.0° C. | 0.187 | 0.277 | 0.326 | 0.26 | 0.07 |
| 15 min Cont 35.0° C. | 0.231 | 0.368 | 0.330 | 0.31 | 0.07 |
| 30 min Cont 35.0° C. | 0.486 | 0.468 | 0.438 | 0.46 | 0.02 |
| 60 min Cont 35.0° C. | 0.427 | 0.688 | 0.725 | 0.61 | 0.16 |
| 120 min Cont 35.0° C. | 0.826 | 0.844 | 0.767 | 0.81 | 0.04 |
| 5 min F21 35.0° C. | 0.553 | 0.388 | 0.318 | 0.42 | 0.12 |
| 15 min F21 35.0° C. | 0.679 | 0.958 | 0.496 | 0.71 | 0.23 |
| 30 min F21 35.0° C. | 1.092 | 0.764 | 1.156 | 1.00 | 0.21 |
| 60 min F21 35.0° C. | 1.620 | 2.278 | 1.307 | 1.74 | 0.50 |
| 120 min F21 35.0° C. | 1.997 | 2.682 | 1.341 | 2.01 | 0.67 |

^{a}Cont = Control = LTS, 0.1% (formula TK1 as described for Batch C)

### Example 14

### Preparation of Formulations CUF-1 (LTS, 0.3% Active Solution) and CUG-1 (LTS, 0.3% Inactive Solution)

Example 7 described the development of a two-component formulation system. The first component is the photosensitizer component comprising lemuteporfin dissolved in a solvent in which it is most soluble. The second component is the diluent component comprising the remainder of the LTS excipients. This example illustrates another two-component lemuteporfin photosensitizer solution (Table 38) with the matching vehicle (placebo) formulation (Table 39). Tables 40 and 41 show two configurations of the two-vial system that yield the same final constituted solution. These formulations simplify the excipients used and were prepared as described above. The new excipient combination meets FDA standards. Further, the total volume of the combined two-component system is reduced in this example for ease of administration in the clinical setting.

TABLE 38
LTS, 0.3% Constituted Solution

| | LTS, 0.3% per vial | |
|---|---|---|
| | grams | wt % |
| Lemuteporfin | 0.009 | 0.3% |
| Benzyl alcohol | 1.491 | 49.7% |
| Isopropyl alcohol | 0.990 | 33.0% |
| DGME | 0.510 | 17.0% |
| Total | 3.00 | 100.00% |

TABLE 39
LTS, 0.3% Vehicle

| | Diluent | Diluent |
|---|---|---|
| | (PCTK-1) | (PCTK-1) |
| | (g) | (% w/w) |
| Lemuteporfin | 0 | 0% |
| Benzyl alcohol | 1.495 | 49.835% |
| Isopropyl alcohol | 0.993 | 33.104% |
| DGME | 0.512 | 17.061% |
| Total | 3.00 | 100.000% |

TABLE 40
2-Vial System for LTS, 0.3%—Configuration 1

| | grams | wt % |
|---|---|---|
| Code | CRE-1 | |
| Identity | Vial 1 (LTS, 0.3% active solution) | |
| Lemuteporfin | 0.009 | 1.00% |
| Benzyl alcohol | 0.891 | 99.00% |
| Total | 0.900 | 100.00% |
| Code | CTK-1 | |
| Identity | Vial 2 (LTS 0.3% inactive solution) | |
| Benzyl alcohol | 0.600 | 28.55% |
| Isopropyl alcohol | 0.990 | 47.15% |
| DGME | 0.510 | 24.30% |
| Total | 2.100 | 100.00% |
| Total Vial 1 + 2 | 3.000 | |

TABLE 41
2-Vial System for LTS, 0.3%—Configuration 2

| | grams | wt % |
|---|---|---|
| Code | CUF-1 | |
| Identity | Vial 1 (LTS, 0.3% active solution) | |
| Lemuteporfin | 0.018 | 0.60% |
| Benzyl alcohol | 2.982 | 99.40% |
| Total | 3.000 | 100.00% |
| Code | CUG-1 | |
| Identity | Vial 2 (LTS 0.3% inactive solution) | |
| Benzyl alcohol | | |
| Isopropyl alcohol | 1.980 | 66.00% |
| DGME | 1.020 | 34.00% |
| Total | 3.000 | 100.00% |
| Total Vial 1 + 2 | 6.000 | |

### Example 15

### Characterization of LTS, 0.3% (Constituted from CUF-1 LTS, 0.3% Active Solution and CUG-1 LTS, 0.3% Inactive Solution)

Vial 1 (LTS, 0.3% Active Solution, code CUF-1), Vial 2 (LTS, 0.3% Inactive Solution, code CUG-1) and the Vehicle (LTS, 0.3% Vehicle, code PCTK-1) were prepared according to Example 14, in order to conduct characterization studies on the new formulation configuration of 6.000-g LTS, 0.3% constituted solution. The specific gravities for these formulations were determined. The chemical and physical stability of the new configuration of LTS, 0.3% was tested by preparing and analyzing filtered and unfiltered samples over a 48-hour time period.

### Determination of Specific Gravity

Before the specific gravity of the samples was determined, the pycnometer was thoroughly cleaned. The weight of the empty pycnometer was then accurately determined. The lid was then removed from the pycnometer and filled with water before it was carefully replaced. After the water extruded out from the overflow orifice, the outer surface of the pycnometer was cleaned and dried. The weight of the pycnometer and water was then accurately measured. Specific gravity testing was performed (N=5) on constituted solution LTS, 0.3%, LTS, 0.3% Vehicle, Formula PCTK-1 and LTS, 0.3% Inactive Solution (Vial 2), Formula CUG-1. The calculations involved in determining the specific gravity of each of the solutions included:
Weight of water=weight of pycnometer with water−weight of pycnometer empty
Weight of sample=weight of pycnometer with sample−weight of pycnometer empty
Specific gravity of sample=(weight of sample)/(weight of water)

### Chemical and Physical Stability of LTS, 0.3% Constituted Solution

LTS, 0.3% Constituted Solution was prepared by adding 3.000-g of Inactive Solution into Vial 1. The entire contents of Vial 1 were transferred using a syringe and needle and either filtered or unfiltered into a new vial at time points: 0, 4, 8, 24 and 48 hours. Triplicate samples were prepared and assayed for each time point. The samples were stored away from light, and the temperature of the dark room was recorded over the 48 hours. After the 48 hours, the samples were sent for chemical analysis. The specific gravity results, based on the data shown in Table 42 were as follows:
LTS, 0.3% Inactive Solution (Vial 2), Formula CUG-1=0.85
LTS, 0.3% Constituted Solution=0.93
LTS, 0.3% Vehicle, Formula PCTK-1=0.94

TABLE 42
Weight of LTS, 0.3% Inactive Solution, Constituted Solution and
Vehicle Samples

| | LTS, 0.3% | LTS, 0.3% | | |
|---|---|---|---|---|
| | Inactive | Consti- | LTS, 0.3% | Weight |
| | Solution, | tuted | Vehicle, | of |
| | Formula | Solution | Formula | Water |
| Sample No. | CUG-1 (g) | (g) | PCTK-1 (g) | (g) |
| 1 | 7.06 | 7.79 | 7.87 | 8.34 |
| 2 | 7.07 | 7.79 | 7.86 | |
| 3 | 7.06 | 7.80 | 7.87 | |
| 4 | 7.07 | 7.80 | 7.88 | |
| 5 | 7.07 | 7.79 | 7.88 | |
| Average | 7.07 | 7.79 | 7.87 | |

### Chemical and Physical Stability

LTS, 0.3% Inactive Solution and Active Solution were prepared as described in Example 14. Vial 1, containing the LTS, 0.3% constituted solution, was either filtered or unfiltered into a new vial over 48 hours. The temperature of the dark room, where the study was conducted, was recorded at multiple times throughout the 48 hours. All samples were stored away from light. The results of these studies is shown in Tables 43, 44 and 45.

TABLE 43
Physical¹ Stability of LTS, 0.3% over 48 Hours following
Constitution

| | | | Lemuteporfin Assay | |
|---|---|---|---|---|
| | Lemuteporfin | | (% Formula | |
| | Assay (% w/w) | | Strength) | |
| Time | Filtered | Unfiltered | Filtered | Unfiltered |
| 0 | 0.29% | 0.29% | 97.4% | 97.7% |
| 4 hours | 0.29% | 0.29% | 96.7% | 97.7% |
| 8 hours | 0.29% | 0.29% | 96.5% | 96.3% |
| 24 hours | 0.29% | 0.29% | 96.9% | 97.4% |
| 48 hours | 0.30% | 0.29% | 99.6% | 96.4% |

¹The physical stability of the supersaturated solution was assessed by filtration.

TABLE 44
Chemical Stability of LTS, 0.3% over 48 Hours following
Constitution

| | | Lemuteporfin | Total Related |
|---|---|---|---|
| | Lemuteporfin | Assay (% Formula | Substances |
| Time | Assay (% w/w) | Strength) | (% Area) |
| 0 | 0.29% | 97.4% | 1.00% |
| 4 hours | 0.29% | 96.7% | 1.01% |
| 8 hours | 0.29% | 96.5% | 0.96% |
| 24 hours | 0.29% | 96.9% | 1.04% |
| 48 hours | 0.30% | 99.6% | 1.14% |

TABLE 45
Weight Loss in LTS, 0.3% Vials over 48 Hours following Constitution

| | | Vial | | | |
|---|---|---|---|---|---|
| | Vial weight | weight at | Weight | Vial weight | Total Weight |
| | at time 0 | 24 Hours | loss at 24 | at 48 | Loss at 48 |
| Vial # | (g) | (g) | hours (%) | Hours (g) | hours (%) |
| 1 | 21.499 | 21.499 | 0.000 | 21.499 | 0.000 |
| 2 | 21.669 | 21.669 | 0.000 | 21.669 | 0.000 |
| 3 | 21.659 | 21.659 | 0.000 | 21.658 | 0.005 |
| 4 | 21.486 | 21.486 | 0.000 | 21.486 | 0.000 |
| 5 | 21.614 | 21.614 | 0.000 | 21.614 | 0.000 |
| 6 | 21.607 | 21.608 | −0.005 | 21.607 | 0.000 |
| 7 | 21.555 | 21.555 | 0.000 | 21.554 | 0.005 |
| 8 | 21.503 | 21.502 | 0.005 | 21.502 | 0.005 |
| 9 | 21.648 | 21.647 | 0.005 | 21.646 | 0.009 |
| 10 | 21.485 | 21.485 | 0.000 | 21.485 | 0.000 |

### CONCLUSION

LTS, 0.3% Inactive Solution (Vial 2), CUG-1, had a specific gravity of 0.85; LTS, 0.3% Constituted Solution, obtained from mixing Vial 1 Active Solution (CUF-1) and Vial 2 Inactive Solution (CUG-1), had a specific gravity of 0.93; and LTS, 0.3% Vehicle, code PCTK-1, had a specific gravity of 0.94. Lemuteporfin content was physically and chemically stable in the new formulation configuration of 6.000-g LTS, 0.3% Constituted Solution. There were no major changes in lemuteporfin content in either filtered or unfiltered samples during the ambient temperature stability study out to 48 hours. Lemuteporfin-related compounds did not change during stability testing.

Having now fully described the inventive subject matter, it will be appreciated by those skilled in the art that the same can be performed within a wide range of equivalent parameters, concentrations, and conditions without departing from the spirit and scope of the disclosure and without undue experimentation. While the disclosure has been described in connection with specific embodiments thereof, it will be understood that it is capable of further modifications. This application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth.

1. A pharmaceutical composition useful for localizing a photosensitizer to a sebaceous gland, comprising a constituted formulation of a photosensitizing component comprising a photosensitizer supersaturated at ambient temperature, one or more solvents, and diethylene glycol monoethyl ether (DGME),
wherein the photosensitizer is lemuteporfin or verteporfin present at a final concentration (w/w) of between about 0.1% to about 0.4% in the pharmaceutical composition; and
wherein the one or more solvents comprise benzyl alcohol present at a final concentration (w/w) of between about 5% and about 55% and isopropanol (IPA) at a final concentration (w/w) of between about 25% to about 60% in the pharmaceutical composition;
wherein the DGME is present at a final concentration (w/w) of about 15% and about 35%; and
wherein the constituted formulation was formed by combining:
a) a first solution of lemuteporfin or verteporfin present in an initial concentration (w/w) of between about 0.5% and 1.5% dissolved in benzyl alcohol; and
b) a second solution of a diluent component comprising DGME present at an initial concentration (w/w) of between about 15% and about 40%, benzyl alcohol present at an initial concentration (w/w) of between about 0% and about 30%, and isopropanol (IPA) present at an initial concentration (w/w) of between about 30% and about 70% in the pharmaceutical composition;
wherein the concentration of the photosensitizer in the constituted formulation is supersaturated at ambient temperature.

2. The pharmaceutical composition of claim 1, wherein the constituted formulation is physically stable for at least 4 hours.

3. The pharmaceutical composition of claim 1, wherein the diluent component optionally additionally comprises oleyl alcohol present at an initial concentration (w/w) of between 4.0% and 6.0%, menthol present at an initial concentration (w/w) of between 2.5% and 3.0%, methyl salicylate present at a final concentration (w/w) of between 0.5% and 1.5%, and polysorbate 80 present at a final concentration (w/w) of between 0.25% and 0.60%.

4. The pharmaceutical composition of claim 1, wherein:
said lemuteporfin is present at a final concentration (w/w) of about 0.3%, said DGME is present at a final concentration (w/w) of about 17%, said benzyl alcohol is present at a final concentration (w/w) of about 49.7%, and said IPA is present at a final concentration (w/w) of about 33%.

5. The pharmaceutical composition of claim 1, wherein:
(a) said first solution comprises lemuteporfin present in an initial concentration (w/w) of about 1.00% and benzyl alcohol at a concentration (w/w) of about 99.00%; and
(b) the diluent component comprises DGME present at an initial concentration (w/w) of about 24.30%, benzyl alcohol present at an initial concentration (w/w) of 28.55%, and IPA present at an initial concentration (w/w) of about 47.15%.

6. The pharmaceutical composition of claim 1, wherein:
(a) said first solution comprises lemuteporfin present in an initial concentration (w/w) of about 0.60% and benzyl alcohol at a concentration (w/w) of about 99.40%; and
(b) the diluent component comprises DGME present at an initial concentration (w/w) of about 34.00%, and IPA present at an initial concentration (w/w) of about 66.00%.

7. The pharmaceutical composition of claim 1, wherein said photosensitizer is lemuteporfin.

8. The pharmaceutical composition of claim 1 for use in a method of treating acne in a subject in need thereof, comprising applying a therapeutically effective amount of said composition to an affected area of the subject's skin having acne lesions, allowing sufficient time for at least some of said lemuteporfin or verteporfin to localize in the sebaceous glands of the affected area, and exposing the skin of the subject to light energy at a wavelength capable of activating said lemuteporfin or verteporfin.

9. The pharmaceutical composition of claim 1 for use in a method for reducing the sebum excretion rate of sebaceous glands in the skin of a subject having an affected area of oily skin, comprising applying a therapeutically effective amount of said pharmaceutical composition to the affected area, allowing sufficient time for at least some of said lemuteporfin or verteporfin to localize in the sebaceous glands, and exposing the skin of the subject to light energy at a wavelength capable of activating the photosensitizer.

10. A method of preparing a pharmaceutical composition of claim 1, comprising mixing a first vial having lemuteporfin or verteporfin and benzyl alcohol, and a second vial having a diluent component comprising diethylene glycol monoethyl ether (DGME) and isopropanol (IPA) and optionally benzyl alcohol, wherein said pharmaceutical composition has a final concentration (w/w) of between about 0.1% to about 0.4% of said lemuteporfin or verteporfin, of between about 5% and about 55% of said benzyl alcohol, of between about 7% and about 25% of said DGME, and of between about 25% and about 60% of said IPA.

11. (canceled)

12. The method of claim 10, wherein:
a) said first vial comprises a solution of lemuteporfin present in an initial concentration (w/w) of about 1.00% and benzyl alcohol at a concentration (w/w) of about 99.00%; and
b) said second vial comprises a solution of DGME present at an initial concentration (w/w) of about 24.30%, benzyl alcohol present at an initial concentration (w/w) of 28.55%, and IPA present at an initial concentration (w/w) of about 47.15%.

13. The method of claim 10, wherein:
a) said first vial comprises a solution of lemuteporfin present in an initial concentration (w/w) of about 0.60% and benzyl alcohol at a concentration (w/w) of about 99.40%; and
b) said second vial comprises a solution of DGME present at an initial concentration (w/w) of about 34.00%, and IPA present at an initial concentration (w/w) of about 66.00%.

14. The method of claim 10, wherein said lemuteporfin or verteporfin is lemuteporfin.

15. A method for reducing the sebum excretion rate of sebaceous glands in the skin of a subject having an area of oily skin, comprising:
(a) applying a therapeutically effective amount of a composition of claim 1 to the affected area on the skin of the subject;
(b) allowing sufficient time for at least some of said lemuteporfin or verteporfin to localize in the sebaceous glands; and
(c) exposing the skin of the subject to light energy at a wavelength capable of activating said lemuteporfin or verteporfin.

16. (canceled)

17. The method of claim 15, wherein said photosensitizer is lemuteporfin.

18. The method of claim 15, wherein the affected area of the subject is pre-treated with dry heat before the composition is applied.

19. The method of claim 15, wherein the time allowed for the photosensitizer to localize is 1 to 2 hours.

20. The method of claim 15, wherein the light energy exposure is in the range of 37.5 to 300 J/cm².

21. A method of treating acne in a subject in need thereof, comprising applying a therapeutically effective amount of a photosensitizer composition of claim 1 to an affected area of the subject's skin having acne lesions, allowing sufficient time for at least some of said lemuteporfin or verteporfin to localize in the sebaceous glands of the affected area, and exposing the skin of the subject to light energy at a wavelength capable of activating the photosensitizer.

22. The method of claim 21, wherein the subject has inflammatory acne lesions, non-inflammatory acne lesions or both inflammatory and non-inflammatory lesions.

23. (canceled)

24. The method of claim 21, wherein said photosensitizer is lemuteporfin.

25. The method of claim 21, wherein the affected area of the subject is pre-treated with dry heat before the composition is applied.

26. The method of claim 21, wherein the time allowed for the photosensitizer to localize is 1 to 2 hours.

27. The method of claim 21, wherein the light energy exposure is in the range of 37.5 to 300 J/cm2.
